SEQUENCE LISTING (30 sequences)

SEQ ID NO: 1 - protein, 110 aa, Artificial Sequence
  VLCD3 Light Chain Variable Domain of Humanized CD123 x CD3 Bispecific Antibody Flotuzumab (DART-A)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 2 - protein, 14 aa, Artificial Sequence
  CDRL1 of VLCD3 Light Chain Variable Domain of Humanized CD123 x CD3 Bispecific Antibody Flotuzumab (DART-A)
RSSTGAVTTSNYAN

SEQ ID NO: 3 - protein, 7 aa, Artificial Sequence
  CDRL2 of VLCD3 Light Chain Variable Domain of Humanized CD123 x CD3 Bispecific Antibody Flotuzumab (DART-A)
GTNKRAP

SEQ ID NO: 4 - protein, 9 aa, Artificial Sequence
  CDRL3 of VLCD3 Light Chain Variable Domain of Humanized CD123 x CD3 Bispecific Antibody Flotuzumab (DART-A)
ALWYSNLWV

SEQ ID NO: 5 - protein, 8 aa, Artificial Sequence
  Linker 1
GGGSGGGG

SEQ ID NO: 6 - protein, 120 aa, Artificial Sequence
  VHCD123 Heavy Chain Variable Domain of Humanized CD123 x CD3 Bispecific Antibody Flotuzumab (DART-A)
EVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSS

SEQ ID NO: 7 - protein, 5 aa, Artificial Sequence
  CDRH1 of VHCD123 Heavy Chain Variable Domain of Humanized CD123 x CD3 Bispecific Antibody Flotuzumab (DART-A)
DYYMK

SEQ ID NO: 8 - protein, 17 aa, Artificial Sequence
  CDRH2 of VHCD123 Heavy Chain Variable Domain of Humanized CD123 x CD3 Bispecific Antibody Flotuzumab (DART-A)
DIIPSNGATFYNQKFKG

SEQ ID NO: 9 - protein, 11 aa, Artificial Sequence
  CDRH3 of VHCD123 Heavy Chain Variable Domain of Humanized CD123 x CD3 Bispecific Antibody Flotuzumab (DART-A)
SHLLRASWFAY

SEQ ID NO: 10 - protein, 113 aa, Artificial Sequence
  VLCD123 Light Chain Variable Domain of Humanized CD123 x CD3 Bispecific Antibody Flotuzumab (DART-A)
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIK

SEQ ID NO: 11 - protein, 17 aa, Artificial Sequence
  CDRL1 of VLCD123 Light Chain Variable Domain of Humanized CD123 x CD3 Bispecific Antibody Flotuzumab (DART-A)
KSSQSLLNSGNQKNYLT

SEQ ID NO: 12 - protein, 7 aa, Artificial Sequence
  CDRL2 of VLCD123 Light Chain Variable Domain of Humanized CD123 x CD3 Bispecific Antibody Flotuzumab (DART-A)
WASTRES

SEQ ID NO: 13 - protein, 9 aa, Artificial Sequence
  CDRL3 of VLCD123 Light Chain Variable Domain of Humanized CD123 x CD3 Bispecific Antibody Flotuzumab (DART-A)
QNDYSYPYT

SEQ ID NO: 14 - protein, 125 aa, Artificial Sequence
  VHCD3 Heavy Chain Variable Domain of Humanized CD123 x CD3 Bispecific Antibody Flotuzumab (DART-A)
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 15 - protein, 5 aa, Artificial Sequence
  CDRH1 of VHCD3 Heavy Chain Variable Domain of Humanized CD123 x CD3 Bispecific Antibody Flotuzumab (DART-A)
TYAMN

SEQ ID NO: 16 - protein, 19 aa, Artificial Sequence
  CDRH2 of VHCD3 Heavy Chain Variable Domain of Humanized CD123 x CD3 Bispecific Antibody Flotuzumab (DART-A)
RIRSKYNNYATYYADSVKD

SEQ ID NO: 17 - protein, 14 aa, Artificial Sequence
  CDRH3 of VHCD3 Heavy Chain Variable Domain of Humanized CD123 x CD3 Bispecific Antibody Flotuzumab (DART-A)
HGNFGNSYVSWFAY

SEQ ID NO: 18 - protein, 6 aa, Artificial Sequence
  Linker 2
GGCGGG

SEQ ID NO: 19 - protein, 28 aa, Artificial Sequence
  “E-Coil” Heterodimer-Promoting Domain
EVAALEKEVAALEKEVAALEKEVAALEK

SEQ ID NO: 20 - protein, 28 aa, Artificial Sequence
  “K-Coil” Heterodimer-Promoting Domain
KVAALKEKVAALKEKVAALKEKVAALKE

SEQ ID NO: 21 - protein, 272 aa, Artificial Sequence
  First Polypeptide Chain of Humanized CD123 x CD3 Bispecific Antibody Flotuzumab (DART-A)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGEVAALEKEVAALEKEVAALEKEVAALEK

SEQ ID NO: 22 - DNA, 816 bp, Artificial Sequence
  Polynucleotide Encoding First Polypeptide Chain of Humanized CD123 x CD3 Bispecific Antibody Flotuzumab (DART-A)
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtgcagtccggggctgagctgaagaaacccggagcttccgtgaaggtgtcttgcaaagccagtggctacaccttcacagactactatatgaagtgggtcaggcaggctccaggacagggactggaatggatcggcgatatcattccttccaacggggccactttctacaatcagaagtttaaaggcagggtgactattaccgtggacaaatcaacaagcactgcttatatggagctgagctccctgcgctctgaagatacagccgtgtactattgtgctcggtcacacctgctgagagccagctggtttgcttattggggacagggcaccctggtgacagtgtcttccggaggatgtggcggtggagaagtggccgcactggagaaagaggttgctgctttggagaaggaggtcgctgcacttgaaaaggaggtcgcagccctggagaaa

SEQ ID NO: 23 - protein, 280 aa, Artificial Sequence
  Second Polypeptide Chain of Humanized CD123 x CD3 Bispecific Antibody Flotuzumab (DART-A)
DFVMTQSPDSLAVSLGERVTMSCKSSQSLLNSGNQKNYLTWYQQKPGQPPKLLIYWASTRESGVPDRFSGSGSGTDFTLTISSLQAEDVAVYYCQNDYSYPYTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGKVAALKEKVAALKEKVAALKEKVAALKE

SEQ ID NO: 24 - DNA, 840 bp, Artificial Sequence
  Polynucleotide Encoding Second Polypeptide Chain of Humanized CD123 x CD3 Bispecific Antibody Flotuzumab (DART-A)
gacttcgtgatgacacagtctcctgatagtctggccgtgagtctgggggagcgggtgactatgtcttgcaagagctcccagtcactgctgaacagcggaaatcagaaaaactatctgacctggtaccagcagaagccaggccagccccctaaactgctgatctattgggcttccaccagggaatctggcgtgcccgacagattcagcggcagcggcagcggcacagattttaccctgacaatttctagtctgcaggccgaggacgtggctgtgtactattgtcagaatgattacagctatccctacactttcggccaggggaccaagctggaaattaaaggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggaaaagtggccgcactgaaggagaaagttgctgctttgaaagagaaggtcgccgcacttaaggaaaaggtcgcagccctgaaagag

SEQ ID NO: 25 - protein, 217 aa, Artificial Sequence
  “Knob-Bearing” CH2 and CH3 Fc Domain
  MISC_FEATURE (217)..(217): XAA is Lys (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGX

SEQ ID NO: 26 - protein, 217 aa, Artificial Sequence
  “Hole-Bearing” CH2 and CH3 Fc Domain
  MISC_FEATURE (217)..(217): XAA is Lys (K) or is Absent
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGX

SEQ ID NO: 27 - protein, 510 aa, Artificial Sequence [sequence omitted]

SEQ ID NO: 28 - protein, 272 aa, Artificial Sequence
  Second Polypeptide Chain of DART-A w/Fc Version 1 Construct
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVQSGAELKKPGASVKVSCKASGYTFTDYYMKWVRQAPGQGLEWIGDIIPSNGATFYNQKFKGRVTITVDKSTSTAYMELSSLRSEDTAVYYCARSHLLRASWFAYWGQGTLVTVSSGGCGGGKVAALKEKVAALKEKVAALKEKVAALKE

SEQ ID NO: 29 - protein, 10 aa, Artificial Sequence
  Peptide 1
DKTHTCPPCP

SEQ ID NO: 30 - protein, 227 aa, Artificial Sequence
  Polypeptide Containing Peptide 1 (SEQ ID NO29) and “Hole-Bearing” CH2 and CH3 Fc Domain (SEQ ID NO26)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

(19) United States
(12) Patent Application Publication
(10) Pub. No.: US 2023/0220087 A1
(43) Pub. Date: Jul. 13, 2023

(54) Use of Bispecific CD123 x CD3 Diabodies for the Treatment of Hematologic Malignancies

(71) Applicants: MacroGenics, Inc., Rockville, MD (US); Nottingham Trent University, Nottingham (GB)

(72) Inventors: Jan Kenneth Davidson, Rockville, MD (US); Sergio Rutella, Nottingham (GB)

(73) Assignees: MacroGenics, Inc., Rockville, MD (US); Nottingham Trent University, Nottingham (GB)

(21) Appl. No.: 18/001,025

(22) PCT Filed: Jun. 9, 2021
(86) PCT No.: PCT/US2021/036520

§ 371 Date: Dec. 7, 2022

Related U.S. Application Data
(60) Provisional application No. 63/041,051, filed on Jun. 18, 2020.

(51) Int. Cl. C07K 16/28; G01N 33/574; C12Q 1/6886

(52) U.S. Cl. CPC C07K 16/2866; C07K 16/2809; G01N 33/57426; C12Q 1/6886; C07K 2317/31; C07K 2317/622; C12Q 2600/158; G01N 2333/7051; G01N 2333/70596

(57) ABSTRACT

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of said hematologic malignancy in said patient. The present invention is particularly directed to the embodiment of such method in which a cellular sample (Z from the patient prior to such administration evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

### REFERENCE TO SEQUENCE LISTING

This application includes one or more Sequence Listings pursuant to 37 C.F.R. 1.821 et seq., which are disclosed in computer-readable media (file name: 1301_0167P1_ST25.txt, created on Jun. 17, 2020, and having a size of 31,062 bytes), which file is herein incorporated by reference in its entirety.

### FIELD OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of said hematologic malignancy in said patient. The present invention is particularly directed to the embodiment of such method in which a cellular sample from the patient prior to such administration evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

### BACKGROUND OF THE INVENTION

I. CD123

CD123 (interleukin 3 receptor alpha, IL-3Ra) is a 40 kDa molecule and is part of the interleukin 3 receptor complex (Stomski, F. C. et al. (1996) “Human Interleukin-3 (IL-3) Induces Disulfide-Linked IL-3 Receptor Alpha- And Beta-Chain Heterodimerization, Which Is Required For Receptor Activation But Not High-Affinity Binding,” Mol. Cell. Biol. 16(6):3035-3046). Interleukin 3 (IL-3) drives early differentiation of multipotent stem cells into cells of the erythroid, myeloid and lymphoid progenitors. CD123 is expressed on CD34+ committed progenitors (Taussig, D. C. et al. (2005) “Hematopoietic Stem Cells Express Multiple Myeloid Markers: Implications For The Origin And Targeted Therapy Of Acute Myeloid Leukemia,” Blood 106:4086-4092), but not by CD34+/CD38− normal hematopoietic stem cells. CD123 is expressed by basophils, mast cells, plasmacytoid dendritic cells, some expression by monocytes, macrophages and eosinophils, and low or no expression by neutrophils and megakaryocytes. Some non-hematopoietic tissues (placenta, Leydig cells of the testis, certain brain cell elements and some endothelial cells) express CD123; however, expression is mostly cytoplasmic.

CD123 is reported to be expressed by leukemic blasts and leukemia stem cells (LSC) (Jordan, C. T. et al. (2000) “The Interleukin-3 Receptor Alpha Chain Is A Unique Marker For Human Acute Myelogenous Leukemia Stem Cells,” Leukemia 14:1777-1784; Jin, W. et al. (2009) “Regulation Of Th17 Cell Differentiation And EAE Induction By MAP3K NIK,” Blood 113:6603-6610). In human normal precursor populations, CD123 is expressed by a subset of hematopoietic progenitor cells (HPC) but not by normal hematopoietic stem cells (HSC). CD123 is also expressed by plasmacytoid dendritic cells (pDC) and basophils, and, to a lesser extent, monocytes and eosinophils (Lopez, A. F. et al. (1989) “Reciprocal Inhibition Of Binding Between Interleukin 3 And Granulocyte-Macrophage Colony-Stimulating Factor To Human Eosinophils,” Proc. Natl. Acad. Sci. (U.S.A.) 86:7022-7026; Sun, Q. et al. (1996) “Monoclonal Antibody 7G3 Recognizes The N-Terminal Domain Of The Human Interleukin-3 (IL-3) Receptor Alpha Chain And Functions As A Specific IL-3 Receptor Antagonist,” Blood 87:83-92; Munoz, L. et al. (2001) “Interleukin-3 Receptor Alpha Chain (CD123) Is Widely Expressed In Hematologic Malignancies,” Haematologica 86(12):1261-1269; Masten, B. J. et al. (2006) “Characterization Of Myeloid And Plasmacytoid Dendritic Cells In Human Lung,” J. Immunol. 177:7784-7793; Korpelainen, E. I. et al. (1995) “Interferon-Gamma Upregulates Interleukin-3 (IL-3) Receptor Expression In Human Endothelial Cells And Synergizes With IL-3 In Stimulating Major Histocompatibility Complex Class II Expression And Cytokine Production,” Blood 86:176-182).

CD123 has been reported to be overexpressed on malignant cells in a wide range of hematologic malignancies including acute myeloid leukemia (AML) and myelodysplastic syndrome (MDS) (Munoz, L. et al. (2001) “Interleukin-3 Receptor Alpha Chain (CD123) Is Widely Expressed In Hematologic Malignancies,” Haematologica 86(12):1261-1269). Overexpression of CD123 is associated with poorer prognosis in AML (Tettamanti, M. S. et al. (2013) “Targeting Of Acute Myeloid Leukaemia By Cytokine-Induced Killer Cells Redirected With A Novel CD123-Specific Chimeric Antigen Receptor,” Br. J. Haematol. 161:389-401).

II. CD3

CD3 is a T cell co-receptor composed of four distinct chains (Wucherpfennig, K. W. et al. (2010) “Structural Biology Of The T-Cell Receptor: Insights Into Receptor Assembly, Ligand Recognition, And Initiation Of Signaling,” Cold Spring Harb. Perspect. Biol. 2(4):a005140; pages 1-14). In mammals, the complex contains a CD3γchain, a CD3δ chain, and two CD3ε chains. These chains associate with a molecule known as the T cell receptor (TCR) in order to generate an activation signal in T lymphocytes. In the absence of CD3, TCRs do not assemble properly and are degraded (Thomas, S. et al. (2010) “Molecular Immunology Lessons From Therapeutic T-Cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th Ed., Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γHeterodimer,” Cell 105(7):913-923; Kuhns, M. S. et al. (2006) “Deconstructing The Form And Function Of The TCR CD3 Complex,” Immunity. 2006 Feb.;24(2):133-139).

III. AML and MDS

Acute myeloid leukemia (AML) and myelodysplastic syndrome (MDS) are thought to arise in, and be perpetuated by, a small population of leukemic stem cells (LSCs), which are generally dormant (i.e., not rapidly dividing cells) and therefore resist cell death (apoptosis) and conventional chemotherapeutic agents. LSCs are characterized by high levels of CD123 expression, which is not present in the corresponding normal hematopoietic stem cell population in normal human bone marrow (Jin, W. et al. (2009) “Regulation Of ThI7 Cell Differentiation And EAE Induction By MAP3K NIK,” Blood 113:6603-6610; Jordan, C. T. et al. (2000) “The Interleukin-3 Receptor Alpha Chain Is A Unique Marker For Human Acute Myelogenous Leukemia Stem Cells,” Leukemia 14:1777-1784). CD123 is expressed in 45%-95% of AML, 85% of Hairy cell leukemia (HCL), and 40% of acute B lymphoblastic leukemia (B-ALL). CD123 expression is also associated with multiple other malignancies/pre-malignancies: chronic myeloid leukemia (CML) progenitor cells (including blast crisis CML); Hodgkin's Reed Sternberg (RS) cells; transformed non-Hodgkin's lymphoma (NHL); some chronic lymphocytic leukemia (CLL) (CD11c+); a subset of acute T lymphoblastic leukemia (T-ALL) (16%, most immature, mostly adult), plasmacytoid dendritic cell (pDC) DC2 malignancies and CD34+/CD38− myelodysplastic syndrome (MDS) marrow cell malignancies.

AML is a clonal disease characterized by the proliferation and accumulation of transformed myeloid progenitor cells in the bone marrow, which ultimately leads to hematopoietic failure. The incidence of AML increases with age, and older patients typically have worse treatment outcomes than younger patients (Robak, T. et al. (2009) “Current And Emerging Therapies For Acute Myeloid Leukemia,” Clin. Ther. 2:2349-2370). Unfortunately, at present, most adults with AML die from their disease.

Treatment for AML initially focuses in the induction of remission (induction therapy). Once remission is achieved, treatment shifts to focus on securing such remission (post-remission or consolidation therapy) and, in some instances, maintenance therapy. The standard remission induction paradigm for AML is chemotherapy with an anthracycline/cytarabine combination, followed by either consolidation chemotherapy (usually with higher doses of the same drugs as were used during the induction period) or human stem cell transplantation, depending on the patient's ability to tolerate intensive treatment and the likelihood of cure with chemotherapy alone (see, e.g., Roboz, G. J. (2012) “Current Treatment Of Acute Myeloid Leukemia,” Curr. Opin. Oncol. 24:711-719).

Agents frequently used in induction therapy include cytarabine and the anthracyclines. Cytarabine (also known as AraC) kills cancer cells (and other rapidly dividing normal cells) by interfering with DNA synthesis. Side effects associated with AraC treatment include decreased resistance to infection, a result of decreased white blood cell production; bleeding, as a result of decreased platelet production; and anemia, due to a potential reduction in red blood cells. Other side effects include nausea and vomiting. Anthracyclines (e.g., daunorubicin, doxorubicin, and idarubicin) have several modes of action including inhibition of DNA and RNA synthesis, disruption of higher order structures of DNA, and production of cell damaging free oxygen radicals. The most consequential adverse effect of anthracyclines is cardiotoxicity, which considerably limits administered life-time dose and to some extent their usefulness.

Stem cell transplantation has been established as the most effective form of anti-leukemic therapy in patients with AML in first or subsequent remission (Roboz, G. J. (2012) “Current Treatment Of Acute Myeloid Leukemia,” Curr. Opin. Oncol. 24:711-719). However, unfortunately, despite substantial progress in the treatment of newly diagnosed AML, 20% to 40% of patients do not achieve remission with the standard induction chemotherapy, and 50% to 70% of patients entering a first complete remission are expected to relapse within 3 years. The optimum strategy at the time of relapse, or for patients with the resistant disease, remains uncertain (see, Tasian, S. K. (2018 “Acute Myeloid Leukemia Chimeric Antigen Receptor T-Cell Immunotherapy: How Far Up The Road Have We Traveled?,” Ther. Adv. Hematol. 9(6):135-148; Przespolewski, A. et al. (2018) “Advances In Immunotherapy For Acute Myeloid Leukemia” Future Oncol. 14(10):963-978; Shimabukuro-Vornhagen, A. et al. (2018) “Cytokine Release Syndrome,” J. Immunother. Cancer. 6(1):56 pp. 1-14; Milone, M. C. et al. (2018) “The Pharmacology of T Cell Therapies,” Mol. Ther. Methods Clin. Dev. 8:210-221; Dhodapkar, M. V. et al. (2017) “Hematologic Malignancies: Plasma Cell Disorders,” Am. Soc. Clin. Oncol. Educ. Book. 37:561-568; Kroschinsky, F. et al. (2017) “New Drugs, New Toxicities: Severe Side Effects Of Modern Targeted And Immunotherapy Of Cancer And Their Management,” Crit. Care 14; 21(1):89). Thus, novel therapeutic strategies are needed.

IV. Bispecific Molecules

The provision of non-monospecific molecules (e.g., bispecific antibodies, bispecific diabodies, BiTE® antibodies, etc.) provides a significant advantage over monospecific molecules such as natural antibodies: the capacity to co-ligate and co-localize cells that express different epitopes. Bispecific molecules thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Of particular importance is the co-ligating of differing cells, for example, the cross-linking of effector cells, such as cytotoxic T cells, to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T-Cells Mediated By A Bispecific Diabody,” Protein Eng. 9:299-305).

In order to provide molecules having greater capability than natural antibodies, a wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further binding protein (e.g., an scFv, VL, VH, etc.) to, or within, the antibody core (IgA, IgD, IgE, IgG or IgM), or to fuse multiple antibody binding portions (e.g., two Fab fragments or scFvs) to one another. Alternative formats use linker peptides to fuse a binding protein (e.g., an scFv, VL, VH, etc.) to a dimerization domain, such as the CH2-CH3 Domain, or to alternative polypeptides (WO 2005/070966, WO 2006/107786 WO 2006/107617, WO 2007/046893) and other formats in which the CL and CH1 Domains are switched from their respective natural positions and/or the VL and VH Domains have been diversified (WO 2008/027236; WO 2010/108127) to allow them to bind to more than one antigen.

The art has additionally noted the capability to produce diabodies that are capable of binding two or more different epitope species (see, e.g., Holliger et al. (1993) “‘Diabodies’: Small Bivalent And Bispecific Antibody Fragments,” Proc. Natl. Acad. Sci. (U.S.A.) 90:6444-6448. Stable, covalently bonded heterodimeric non-monospecific diabodies have been described (see, e.g., WO 2006/113665; WO/2008/157379; WO 2010/080538; WO 2012/018687; WO/2012/162068; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Moore, P. A. et al. (2011) “Application Of Dual Affinity Retargeting Molecules To Achieve Optimal Redirected T-Cell Killing Of B-Cell Lymphoma,” Blood 117(17):4542-4551). Such diabodies incorporate one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule. In addition, trivalent molecules comprising a diabody-like domain have been described (see, e.g., WO 2015/184203; and WO 2015/184207). Diabody epitope binding domains may also be directed to a surface determinant of any immune effector cell such as CD3, CD16, CD32, or CD64, which are expressed on T lymphocytes, natural killer (NK) cells or other mononuclear cells. In many studies, diabody binding to effector cell determinants, e.g., Fcγ receptors (FcγR), was also found to activate the effector cell (Holliger et al. (1996) “Specific Killing Of Lymphoma Cells By Cytotoxic T-Cells Mediated By A Bispecific Diabody,” Protein Eng. 9:299-305; Holliger et al. (1999) “Carcinoembryonic Antigen (CEA)—Specific T-cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen-bound antibody to an effector cell via Fc-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of the tumor cell, but leads to effective tumor killing (see e.g., Cao et al. (2003) “Bispecific Antibody Conjugates In Therapeutics,” Adv. Drug. Deliv. Rev. 55:171-197).

Several bispecific molecules targeting CD123 and CD3 capable of mediating T cell redirected cell killing of CD123-expressing malignant cells are in development (see, e.g., Vey, N., et al. (2017) “Interim Results From A Phase I First-In-Human Study Of Flotetuzumab, a CD123× CD3 Bispecific DART Molecule In AML/MDS,” Annals of Oncology, 28(S5)5, mdx373.001; Godwin, C. D., et al. (2017) “Bispecific Anti-CD123×Anti-CD3 Adaptir™ Molecules APVO436 and APVO437 Have Broad Activity Against Primary Human AML Cells In Vitro” Blood. 130(S1): 2639; Forslund, A., et al. (2016) “Ex Vivo Activity Profile of the CD123xCD3 Duobody® Antibody JNJ—63709178 Against Primary Acute Myeloid Leukemia Bone Marrow Samples” Blood 128(22):2875). However, efforts to employ bispecific binding molecules that are capable of targeting a T cell to the location of a hematologic malignancy have not been fully successful. Hence, an unmet need remains to develop new strategies for the treatment of hematologic malignancies with CD123×CD3 bispecific binding molecules. The present invention directly addresses this need and others, as described below.

### SUMMARY OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of the hematologic malignancy in the patient. The present invention is particularly directed to the embodiment of such method in which a cellular sample from the patient prior to such administration evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

In detail, the invention provides a method of determining whether a patient would be a suitable responder to the use of a CD123×CD3 bispecific molecule to treat a hematologic malignancy, wherein the method comprises:
(a) evaluating the expression of one or more target genes in a cellular sample from the patient prior to the administration of the CD123×CD3 bispecific molecule, relative to the expression of one or more target and/or reference genes; and
(b) identifying the patient as a suitable responder for treatment with a CD123×CD3 bispecific molecule if the expression of the one or more target genes is found to be increased relative to the expression of the one or more target and/or reference genes, wherein said one or more target genes are selected from the group consisting of: SERPHINH1, NOTCH2, FCGR3A/B, FPR1, FBP1, PDGFA, CRABP2, THBS1, ICOS and CD8B.

The invention further provides the embodiment of such methods wherein the method evaluates: (i) the expression of one or more target gene; and (ii) one or more reference gene whose expression is not characteristically associated with the hematologic malignancy.

The invention further provides the embodiment of such methods that comprises evaluating the expression of the one or more target genes relative to the baseline expression of the one or more reference genes of the patient.

The invention further provides the embodiment of such methods that comprises evaluating the expression of the one or more target genes of a patient relative to the expression of the one or more target genes of an individual who is suffering from the hematologic malignancy or of a population of such individuals. The invention further provides the embodiment of such methods wherein the expression of the one or more target genes of such patient is greater than the first quartile (i.e., greater than the bottom 25%), greater than the second quartile (i.e., greater than the bottom 50%), or greater than the third quartile (i.e., greater than the bottom 75%) of the expression levels of such target gene(s) of such individual or of such population of individuals who are suffering from the hematologic malignancy.

The invention further provides the embodiment of such methods that comprises evaluating the expression of the one or more target genes of a patient relative to the expression of the one or more target genes of an individual who had previously been unsuccessfully treated for a hematologic malignancy using the methods and compositions of the present invention (e.g., an individual who did not successfully respond to a treatment for a hematologic malignancy using a CD123×CD3 bispecific molecule), or a population of such individuals. The invention further provides the embodiment of such methods wherein the expression of the one or more target genes of such patient is greater than the first quartile (i.e., greater than the bottom 25%), greater than the second quartile (i.e., greater than the bottom 50%), or greater than the third quartile (i.e., greater than the bottom 75%) of the expression levels of such target gene(s) of such individual or of such population of unsuccessfully treated individuals.

The invention further provides the embodiment of such methods that comprises evaluating the expression of the one or more target genes of a patient relative to the expression of the one or more target genes of an individual who had previously been successfully treated for a hematologic malignancy using the methods and compositions of the present invention (e.g., an individual who successfully responded to a treatment for a hematologic malignancy using a CD123×CD3 bispecific molecule) or a population of such individuals. The invention further provides the embodiment of such methods wherein the expression of the one or more target genes of such patient is within the first quartile (i.e., within the bottom 25%) of the expression levels of such target gene(s), within the second quartile (i.e., between the bottom 25% and 50%), or within the third quartile (i.e., between the bottom 50% and 75%) of the expression levels of such target gene(s) of such individual or such population of successfully treated individuals.

The invention further provides the embodiment of such methods wherein the relative expression level of the one or more target genes in the population is established by averaging the gene expression level in cellular samples obtained from the population of individuals.

The invention further provides the embodiment of such methods wherein such patient exhibits an expression level of at least one of such target genes:
(a) that is greater than the first quartile of the expression levels of such target gene in a population of individuals who are suffering from the hematologic malignancy; or
(b) that is greater than the first quartile of the expression levels of such target gene in a population of individuals who did not successfully respond to a treatment for the hematologic malignancy that used a CD123×CD3 bispecific molecule; or
(c) that is within at least the first quartile of the expression levels of such target gene in a population of individuals who successfully responded to a treatment for a hematologic malignancy that used a CD123×CD3 bispecific molecule.

The invention further provides the embodiment of such methods wherein such patient exhibits an expression level of at least one of such target genes:
(a) that is greater than the second quartile of the expression levels of such target gene in a population of individuals who are suffering from the hematologic malignancy; or
(b) that is greater than the second quartile of the expression levels of such target gene in a population of individuals who did not successfully respond to a treatment for the hematologic malignancy that used a CD123×CD3 bispecific molecule; or
(c) that is within at least the second quartile of the expression levels of such target gene in a population of individuals who successfully responded to a treatment for the hematologic malignancy that used a CD123×CD3 bispecific molecule.

The invention further provides the embodiment of such methods wherein such patient exhibits an expression level of at least one of such target genes:
(a) that is greater than the third quartile of the expression levels of such target gene in a population of individuals who are suffering from said hematologic malignancy; or
(b) that is greater than the third quartile of the expression levels of such target gene in a population of individuals who did not successfully respond to a treatment for said hematologic malignancy that used a CD123×CD3 bispecific molecule; The invention further provides the embodiment of such methods wherein such method further comprises administering a treatment dosage of the CD123×CD3 bispecific molecule to the patient if the patient is determined to be a suitable responder to such treatment, and to such methods wherein the administration of the CD123×CD3 bispecific molecule stimulates the killing of cells of the hematologic malignancy in the patient.

The invention further provides a method of treating a hematologic malignancy, wherein the method comprises:
(a) employing the method of any one of the above embodiments to determine whether a patient would be a suitable responder to the use of a CD123×CD3 bispecific molecule to treat the hematologic malignancy;
(b) administering a treatment dosage of the CD123×CD3 bispecific molecule to the patient if the patient is determined to be a suitable responder to such treatment;
   wherein the administration of the CD123×CD3 bispecific molecule stimulates the killing of cells of the hematologic malignancy in the patient.

The invention further provides the embodiment of such methods that additionally comprises evaluating the expression of such one or more target genes in a cellular sample obtained from the patient one or more times after the initiation of the treatment.

The invention further provides the embodiment of such methods wherein the cellular sample is a bone marrow or a blood sample. Particularly, the embodiment of such methods wherein the cellular sample is a bone marrow sample.

The invention further provides the embodiment of such methods that further comprises detecting the expression level of one or more target genes in a sample of the patient's bone marrow. The invention further provides the embodiment of such methods that further comprises detecting the expression level of one or more reference genes.

The invention further provides the embodiment of such methods that comprise detecting the expression level of such one or more target genes and/or such one or more reference genes in a sample of the patient's bone marrow, particularly prior to administration of a CD123×CD3 bispecific molecule.

The invention further provides the embodiment of such methods wherein the evaluation of expression or the determination of whether the patient would be a suitable responder to the use of a CD123×CD3 bispecific molecule to treat a hematologic malignancy is performed by:
(a) determining the gene expression levels for each target gene in one or more cellular sample(s) using a gene expression platform; and
(b) comparing the target gene expression levels to the expression levels of one or more reference genes.

The invention further provides the embodiment of such methods wherein the evaluation of expression or the determination of whether the patient would be a suitable responder to the use of a CD123×CD3 bispecific molecule to treat a hematologic malignancy is performed by:
(a) measuring the raw RNA levels for each target gene in one or more cellular sample(s) in a gene expression platform;
wherein the gene expression platform comprises a reference gene set of housekeeping genes; and
(b) assigning a relative expression value, for each of the measured raw RNA levels for the target genes using the measured RNA levels of the internal reference genes.

The invention further provides the embodiment of such methods wherein the one or more reference genes comprise one or more of: ABCF1, G6PD, NRDE2, OAZ1, POLR2A, SDHA, STK11IP, TBC1D10B, TBP, and UBB.

The invention further provides the embodiment of such methods wherein a gene signature score is determined for the one or more target genes. In specific embodiments of the invention such gene signature score is determined from the raw RNA levels of each target gene by a process comprising:
(a) measuring the raw RNA levels for each target gene in one more cellular sample using a gene expression platform comprising a reference gene set of housekeeping genes,
(b) normalizing each of the measured raw RNA levels to the geometric mean of such housekeeping genes, and optionally further normalizing each RNA value to a standard,
(c) log transforming each normalized RNA value,
(d) summing the log transformed RNA values for each target gene in the signature, and
(e) dividing the sum of the normalized log transformed RNA values by the number of target genes in the signature, to generate a gene signature score.

The invention further provides the embodiment of such methods wherein a patient gene signature score that:
(a) is greater than the first quartile of scores for the gene signature calculated from the expression levels of one or more of the target genes in a population of individuals who are suffering from the hematologic malignancy; or
(b) is greater than the first quartile of scores for the gene signature calculated from the expression levels of one or more of the target genes in a population of individuals who did not successfully respond to a treatment for the hematologic malignancy that used a CD123×CD3 bispecific molecule; or
(c) is within at least the first quartile of the scores for the gene signature calculated from the expression levels of one or more of the target genes in a population of individuals who successfully responded to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein a patient gene signature score that:
(a) is greater than the second quartile for the gene signature calculated from the expression levels of one or more of the target genes in a population of individuals who are suffering from the hematologic malignancy; or
(b) is greater than the second quartile for the gene signature calculated from the expression levels of one or more of the target genes in a population of individuals who did not successfully respond to a treatment for the hematologic malignancy that used a CD123×CD3 bispecific molecule; or
(c) is within at least the second quartile of the scores for the gene signature calculated from the expression levels of one or more of the target genes in a population of individuals who successfully responded to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein a patient gene signature score that:
(a) is greater than the third quartile of scores for the gene signature calculated from the expression levels of one or more of the target genes in a population of individuals who are suffering from the hematologic malignancy; or
(b) is greater than the third quartile of scores for the gene signature calculated from the expression levels of one or more of the target genes in a population of individuals who did not successfully respond to a treatment for the hematologic malignancy that used a CD123×CD3 bispecific molecule,
   is indicative of a more favorable patient response to treatment with the CD123×CD3 bispecific molecule.

The invention further provides the embodiment of such methods wherein the CD123×CD3 bispecific molecule is a bispecific antibody or a bispecific molecule comprising an scFv.

The invention further provides the embodiment of such methods wherein the CD123×CD3 bispecific molecule is JNJ-63709178, XmAb14045 or APVO436.

The invention further provides the embodiment of such methods wherein the CD123×CD3 bispecific molecule is a covalently bonded bispecific diabody having two, three, or four polypeptide chains.

The invention further provides the embodiment of such methods wherein the CD123×CD3 bispecific molecule comprises:
(a) a VH_{CD123} Domain comprising the CDRs of SEQ ID NO:6; and
(b) a VL_{CD123} Domain comprising the CDRs of SEQ ID NO:10.

The invention further provides the embodiment of such methods wherein the CD123×CD3 bispecific molecule comprises:
(a) a VH_{CD123} Domain comprising SEQ ID NO:6; and
(b) a VL_{CD123} Domain comprising SEQ ID NO:10.

The invention further provides the embodiment of such methods wherein the CD123×CD3 bispecific molecule comprises:
(a) a VH_{CD3} Domain comprising the CDRs of SEQ ID NO:14; and
(b) a VL_{C23} Domain comprising the CDRs of SEQ ID NO:1.

The invention further provides the embodiment of such methods wherein the CD123×CD3 bispecific molecule comprises:
(a) a VH_{CD3} Domain comprising SEQ ID NO:14; and
(b) a VL_{CD3} Domain comprising SEQ ID NO:1.

The invention further provides the embodiment of such methods wherein the CD123×CD3 bispecific molecule is a diabody that comprises:
(a) a first polypeptide chain comprising the amino acid sequence of SEQ ID NO:21; and
(b) a second polypeptide chain comprising the amino acid sequence of SEQ ID NO:23; and
   wherein the first and the second polypeptide chains are covalently bonded to one another by a disulfide bond.

The invention further provides the embodiment of such methods wherein the hematologic malignancy of such patient is selected from the group consisting of: acute myeloid leukemia (AML), chronic myelogenous leukemia (C-L), blastic crisis of CML, Abelson oncogene-associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), Richter's syndrome, Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

The invention further provides the embodiments of such methods wherein the hematologic malignancy of such patient is AML, MDS, BPDCN, or T-ALL.

The invention further provides the embodiment of such methods wherein the hematologic malignancy of such patient is refractory to chemotherapy (CTX), such as being refractory to cytarabine/anthracycline-based cytotoxic chemotherapy or refractory to hypomethylating agents (HMA) chemotherapy.

The invention further provides the embodiment of such methods that further comprises determining the level expression of CD123 of blast cells (cancer cells) as compared to a corresponding baseline level CD123 expressed by normal peripheral blood mononuclear cells (PBMCs).

The invention further provides the embodiment of such methods wherein the level of expression is determined by measuring the cell surface expression of CD123. The invention further provides the embodiment of such methods wherein the cell surface expression of CD123 is increased by at least about 20% relative to a baseline level of expression. The invention further provides the embodiment of such methods wherein the increase in CD123 expression renders the patient more responsive to treatment with the CD123×CD3 bispecific molecule.

The invention further provides the embodiment of such methods wherein the effective dosage of the CD123×CD3 bispecific molecule is selected from the group consisting of about 30, about 60, about 100, about 200, about 300, about 400, and about 500 ng/kg patient weight/day.

The invention further provides the embodiment of all of the above-described methods wherein the treatment dosage is administered as a continuous infusion. The invention further provides the embodiment of such methods wherein the treatment dosage is about 30 ng/kg/day administered by continuous infusion for 1 day, followed by a treatment dosage of about 60 ng/kg patient weight/day administered by continuous infusion for 1 day, followed by a treatment dosage of about 100 ng/kg/day administered by continuous infusion for 1 day, followed by a treatment dosage of about 200 ng/kg/day administered by continuous infusion for 1 day, followed by a treatment dosage of about 300 ng/kg/day administered by continuous infusion for 1 day, followed by a treatment dosage of about 400 ng/kg/day administered by continuous infusion for 1 day, followed by a treatment dosage of about 500 ng/kg/day administered by continuous infusion for 1 day. The invention further provides the embodiment of such methods wherein the treatment dosage further comprises administration of about 500 ng/kg/day administered by continuous infusion for up to an additional 21 days.

The invention further provides the embodiment of all of the above-described methods wherein the patient is a human patient.

### BRIEF DESCRIPTION OF THE DRAWINGS

FIGS. 1A-1C illustrate the overall structure of exemplary diabody molecules. FIG. 1A provides the structure of the first and second polypeptide chains of a two chain CD123×CD3 bispecific diabody (“DART-A” also known as flotetuzumab) having two epitope-binding domains, Heterodimer-Promoting Domains and a cysteine containing linker. FIGS. 1B-1C provide the overall structure of a CD123×CD3 bispecific diabody having two epitope-binding domains composed of three polypeptide chains. Two of the polypeptide chains possess a CH2 and CH3 Domain, such that the associated chains form all or part of an Fc Domain. The polypeptide chains comprising the VL and VH Domain further comprise a Heterodimer-Promoting Domain and a linker. A cysteine residue may be present in a linker (FIGS. 1A and 1B) and/or in the Heterodimer-Promoting Domain (FIG. 1C). VL and VH Domains that recognize the same epitope are shown using the same shading or fill pattern.

FIG. 2 illustrates the expression of the top 10 genes associated with complete response to flotetuzumab (complete remission (CR), complete remission with partial hematopoietic recovery (CRh), complete remission with incomplete hematopoietic recovery (CRi)). Expression of each gene within this cohort was scaled from −2 to +2. Patient response after treatment, and status and immune cluster at the time of inclusion in the study are indicated in the top rows. The immune cluster status was defined as previously detailed (Vadakekolathu J, et al. (2020) “Immune Landscapes Predict Chemotherapy Resistance And Immunotherapy Response In Acute Myeloid Leukemia,” Sci Transl Med. 12:eaaz0463).

FIG. 3 plots the 10-gene signature scores for patients exhibiting a complete response, a partial response, and no response.

FIG. 4 shows a heatmap summarizing the correlation coefficients between the 10-gene classifier score and immune cell type-specific and biological activity signature scores in baseline bone marrow samples from patients with relapsed refractory AML.

FIG. 5 shows AUROC curves measuring the predictive ability of the 10-gene signature score and the ELN cytogenetic risk, alone or combined for anti-leukemic activity from flotetuzumab.

### DETAILED DESCRIPTION OF THE INVENTION

The present invention is directed to a method of treating a hematologic malignancy such as acute myeloid leukemia (AML) or myelodysplastic syndrome (MDS), including hematologic malignancies that are refractive to chemotherapeutic and/or hypomethylating agents. The method concerns administering a CD123×CD3 bispecific binding molecule to a patient in an amount effective to stimulate the killing of cells of said hematologic malignancy in said patient. The present invention is particularly directed to the embodiment of such method in which a cellular sample from the patient prior to such administration evidences an expression of one or more target genes that is increased relative to a baseline level of expression of such genes, for example, a baseline level of expression of such genes in a reference population of individuals who are suffering from the hematologic malignancy, or with respect to the level of expression of a reference gene.

As indicated above, chemotherapy resistance and relapse remain significant sources of mortality for children and adults with acute myeloid leukemia (AML). Receiving conventional chemotherapy, only 26.9% of patients are expected to survive beyond 5 years.

The therapeutic approach in patients with acute myeloid leukemia (AML) has not changed substantially in more than 30 years. The standard front line therapy is a two-drug regimen of cytarabine given in conjunction with daunorubicin (the so-called 7+3 induction therapy, abbreviated herein as “CTX”). The hypomethylating agents (abbreviated herein as “HMA”) decitabine and azacitidine are commonly administered to older patients or to those considered unfit for the CTX regimen. However, estimates from the literature indicate that up to 45% of patients are refractory to standard frontline chemotherapy. Further intensification of conventional cytotoxic chemotherapy has been deemed to not be feasible due to the severity of acute and long-term side effects upon normal tissues commonly induced by these drugs (Tasian, S. K. (2018 “Acute Myeloid Leukemia Chimeric Antigen Receptor T-Cell Immunotherapy: How Far Up The Road Have We Traveled?,” Ther. Adv. Hematol. 9(6):135-148; Przespolewski, A. et al. (2018) “Advances In Immunotherapy For Acute Myeloid Leukemia” Future Oncol. 14(10):963-978; Shimabukuro-Vornhagen, A. et al. (2018) “Cytokine Release Syndrome,” J. Immunother. Cancer. 6(1):56 pp. 1-14; Milone, M. C. et al. (2018) “The Pharmacology of T Cell Therapies,” Mol. Ther. Methods Clin. Dev. 8:210-221; Dhodapkar, M. V. et al. (2017) “Hematologic Malignancies: Plasma Cell Disorders,” Am. Soc. Clin. Oncol. Educ. Book. 37:561-568; Kroschinsky, F. et al. (2017) “New Drugs, New Toxicities: Severe Side Effects Of Modern Targeted And Immunotherapy Of Cancer And Their Management,” Crit. Care 14; 21(1):89).

Bispecific antibodies that engage T cells stimulate the release of proinflammatory cytokines. Such cytokines can increase anti-leukemia efficacy by direct cytotoxicity and by activation and recruitment of immune cells into the tumor site (Hoseini, S. S. et al. (2107) “Acute Myeloid Leukemia Targets For Bispecific Antibodies,” Blood Cancer Journal 7:e522, doi:10.1038/bcj.2017.2; pp. 1-12. In particular, treatment with flotetuzumab, a CD123×CD3 bispecific binding molecule, is being tested in a Phase ½ study of relapsed/refractory (“R/R”) AML. Despite the great potential of immunotherapy to selectively target the cancer cells causing hematologic malignancies (see, e.g., Koch, J. et al. (2017) “Recombinant Antibodies to Arm Cytotoxic Lymphocytes in Cancer Immunotherapy,” Transfus. Med. Hemother. 44:337-350; Lichtenegger, F. S. et al. (2017) “Recent Developments In Immunotherapy Of Acute Myeloid Leukemia,” J. Hematol. Oncol. 10:142, pp. 1-20), efforts to employ bispecific binding molecules that are capable of targeting a T cell to the location of a hematologic malignancy have not been fully successful.

The discovery of new treatment strategies, including immunotherapy, thus remains a priority. It has previously been reported that AML patients with an immune-enriched and IFN gamma-dominant tumor microenvironment (“TME”) experience significantly shorter relapse-free survival, suggesting refractoriness to standard induction chemotherapy (Vadakekolathu, J. et al. (2017) “Immune Gene Expression Profiling in Children and Adults with Acute Myeloid Leukemia Identifies Distinct Phenotypic Patterns,” Blood 130:3942A). In addition, certain gene expression signatures have been reported to correlate with response to the CD123×CD3 bispecific molecule, flotetuzumab (Vadakekolathu J, et al. (2020) “Immune Landscapes Predict Chemotherapy Resistance And Immunotherapy Response In Acute Myeloid Leukemia,” Sci. Transl. Med. 12(546):eaaz0463).

As used herein, the term “gene expression signature” is intended to denote a pattern of gene expression of a group of genes that is characteristic of a particular cell type and/or biological process (see, e.g., Stenner, F. et al. (2018) “Cancer Immunotherapy and the Immune Response in Follicular Lymphoma,” Front. Oncol. 8:219 doi: 10.3389/fonc.2018.00219, pages 1-7; Cesano, A. et al. (2018) “Bringing The Next Generation Of Immuno-Oncology Biomarkers To The Clinic,” Biomedicines 6(14) doi: 10.3390/biomedicines6010014, pages 1-11; Shrestha, G. et al. (2016) “The Value Of Genomics In Dissecting The RAS-Network And In Guiding Therapeutics For RAS-Driven Cancers,” Semin. Cell Dev. Biol. 58:108-117; Gingras, I. et al. (2015) “CCR 20th Anniversary Commentary: Gene-Expression Signature in Breast Cancer—Where Did It Start and Where Are We Now?,” Clin. Cancer Res. 21(21):4743-4746; Eberhart, C. G. (2011) “Molecular Diagnostics In Embryonal Brain Tumors,” Brain Pathol. 21(1):96-104; Baylin, S. B. (2009) “Stem Cells, Cancer, And Epigenetics,” StemBook, ed. THE STEM CELL RESEARCH COMMUNITY, StemBook, doi/10.3824/stembook.1.50.1, pages 1-14; Asakura, M. et al. (2009) “Global Gene Expression Profiling In The Failing Myocardium,” Circ. J. 73(9):1568-1576; Shaffer, A. L. et al. (2001) “Signatures Of The Immune Response,” Immunity 15(3):375-385; Staudt, L. M. et al. (2005) “The Biology Of Human Lymphoid Malignancies Revealed By Gene Expression Profiling,” Adv. Immunol. 87:163-208). An observed gene expression signature, and/or changes in that signature resulting from altered (or unaltered) biological process(es), can be used to assess the presence, nature and/or severity of a pathogenic medical condition.

A central aspect of the present invention relates to the identification of a unique “10-gene expression signature” that predicts a favorable response to therapy employing CD123×CD3 bispecific binding molecules, including therapy employing the CD123×CD3 bispecific binding molecule, flotetuzumab. The 10 genes of the “10-gene expression signature” are: SERPHINH1, NOTCH2, FCGR3A/B, FPR1, FBP1, PDGFA, CRABP2, THBS1, ICOS and CD8B. The invention derives in part from the recognition that certain sub-populations of patients having a hematologic malignancy (e.g., an acute myeloid leukemia) are particularly amenable to treatment with the CD123×CD3 bispecific binding molecules (e.g., flotetuzumab). Members of this sub-population can be readily identified by their ability to exhibit elevated expression of such 10-gene expression signature.

I. Identification of Patient Populations Particularly Suitable for Treatment with the CD123×CD3 Bispecific Binding Molecules of the Invention

A. Methods for Determining “Gene Expression Signatures”

In order to determine whether a patient exhibits elevated expression of the 10-gene expression signature, so as to be thereby identified as being particularly amenable for the treatment of a hematologic malignancy using the methods and compositions of the present invention, an RNA sample from a cellular sample obtained from a patient is evaluated to determine whether it evidences increased expression of one or more “target” genes whose expression correlates with such a signature. Such evaluation may make use of pre-existing detection and/or measurements of gene expression or may incorporate the step(s) of detecting and/or measuring such gene expression. As used herein, the term “cellular sample” refers to a sample that contains cells or an extract of cells.

Any cellular sample may be employed as a source of RNA or protein for use in determining whether a patient exhibits the 10-gene expression signature that is characteristic of response to a favorable response to therapy employing CD123×CD3 bispecific binding molecules. In certain embodiments, such gene expression comparisons are conducted using RNA obtained from a bone marrow (BM) sample or from a blood sample or a sample of blast cells (cancer cells) of the patient or of a population of donors. Where RNA is obtained from such cells of a population of donors to provide a baseline expression level, the average of the employed expression levels may be used (e.g., a geometric mean may be employed). A number of different reference populations may be used for such gene expression comparisons. In particular embodiments, the expression level of at least one target gene exhibited by a patient is compared to the expression level of such target gene exhibited in: a population of individuals who are suffering from a hematologic malignancy; a population of individuals who were suffering from such hematologic malignancy at the time such reference expression level was determined and who did not successfully respond to a treatment for a hematologic malignancy (i.e., a population of individuals who did not successfully respond to a treatment for a hematologic malignancy using a CD123×CD3 bispecific molecule); and/or a population of individuals who were suffering from such hematologic malignancy at the time such reference expression level was determined and who were thereafter successfully treated for a hematologic malignancy using the methods and compositions of the present invention (i.e., a population of individuals who successfully responded to a treatment for a hematologic malignancy using a CD123×CD3 bispecific molecule). Where the comparator population is a population of individuals who are suffering from a hematologic malignancy such population preferably includes individuals who are suffering from the same hematological malignancy as the patient. Such population may include individuals that have relapsed after prior treatment with a chemotherapeutic agent and/or that were refractory to treatment with a chemotherapeutic agent (i.e., primary refractory). Where the comparator population is a population of individuals who successfully, or unsuccessfully responded to a treatment for a hematologic malignancy CD123×CD3 bispecific molecule such population preferably includes individuals who are suffering from the same hematological malignancy as the patient.

As used herein, the expression of a gene is said to be “increased” if, relative to a baseline or other comparator (e.g., expression of such gene in a population), its expression is at least about 10% greater, at least about 20% greater, at least about 30% greater, at least about 40% greater, at least about 50% greater, at least about 60% greater, at least about 70% greater, at least about 80% greater, at least about 90% greater, at least about 1.5-fold greater, at least about 2-fold greater, at least about 2.5-fold greater, at least about 3-fold greater, at least about 3.5-fold greater, at least about 4-fold greater, at least about 4.5-fold greater, at least about 5-fold greater, at least about 5.5-fold greater, at least about 6-fold greater, at least about 6.5-fold greater, at least about 7-fold greater, at least about 7.5-fold greater, at least about 8-fold greater, at least about 8.5-fold greater, at least about 9-fold greater, at least about 10-fold greater. Such increases can be alternatively described in terms of “log 2-fold changes.” With respect to increases in expression, a log 2-fold change of 0.4 is equivalent to about 30% greater expression a log 2-fold change of 0.5 is equivalent to about 40% greater expression; a log 2-fold change of 0.6 is equivalent to about 50% greater expression; a log 2-fold change of 0.7 is equivalent to about 60% greater expression; a log 2-fold change of 0.8 is equivalent to about 70% greater expression; a log 2-fold change of 0.9 is equivalent to about 90% greater expression; a log 2-fold change of 1 is equivalent to a 2-fold increase; a log 2-fold change of 1.5 is equivalent to a 2.8-fold increase; a log 2-fold change of 2 is equivalent to a 4-fold increase; a log 2-fold change of 2.5 is equivalent to a 5.7-fold increase; a log 2-fold change of 3 is equivalent to an 8-fold increase; a log₂-fold change of 3.5 is equivalent to an 11.3-fold increase; a log 2-fold change of 4 is equivalent to a 16-fold increase, etc. Log₂ fold changes are commonly used when comparing counts to array data and are also appropriate for t-tests.

Alternatively, such increases are described in terms of a “gene signature score” wherein the expression of each of a cluster of target genes is measured, normalized to one or more housekeeping genes and/or internal standards, and summed to generate a single gene signature score. Optionally, after normalization and prior to summing, the expression of each target gene may be log transformed, and/or weighted. Methods for calculating such scores are known in the art and specific methods are provided herein (see, Example 1 below).

The 10-gene signature score of a patient is also said to be “increased” if it is greater than the first quartile of gene signature scores (i.e., greater than the bottom 25%), greater than the second quartile of gene signature scores (i.e., greater than the lower 50%), greater than the third quartile of gene signature scores (i.e., greater than the lower 75%), greater than 85%, greater than 90%, or greater than 95% of the gene signature scores calculated from the expression levels of such target genes in a population of individuals who are suffering from a hematologic malignancy.

The 10-gene signature score of a patient is also said to be “increased” if it is greater than the first quartile of gene signature scores (i.e., greater than the bottom 25%), greater than the second quartile of gene signature scores (i.e., greater than the lower 50%), greater than the third quartile of gene signature scores (i.e., greater than the lower 75%), greater than 85%, greater than 90%, or greater than 95% of the gene signature scores calculated from the expression levels of such target genes in a population of individuals who did not successfully respond to a treatment for a hematologic malignancy (e.g., a population of individuals who did not successfully respond to a treatment for a hematologic malignancy CD123×CD3 bispecific molecule).

The 10-gene signature score of a patient is also said to be “increased” if it is within at least the first quartile of gene signature scores (i.e., within the bottom 25%), within at least the second quartile (i.e., between the bottom 25% and 50%), within at least the third quartile (i.e., between the bottom 50% and 75%), greater than 85%, greater than 90%, or greater than 95% of the gene signature scores calculated from the expression levels of such target genes in a population of individuals who have previously been successfully treated for a hematologic malignancy using the methods and compositions of the present invention (e.g., a population of individuals who successfully responded to a treatment for a hematologic malignancy using a CD123×CD3 bispecific molecule).

A finding of an increased 10-gene signature score is indicative of a more favorable patient response to treatment for hematologic malignancy with the CD123×CD3 bispecific molecules of the present invention.

In one embodiment, a patient is identified as exhibiting an elevated 10-gene expression signature and to thus be particularly amenable to the treatment of hematologic malignancy using the methods and compositions of the present invention by determining whether the expression of a target gene is “increased” relative to the baseline level of its expression in the patient being evaluated when such patient was healthy, or before such patient had received a diagnosis of hematologic malignancy, or relative to the expression of that gene at a time during such patient's course of a chemotherapy treatment regimen or during such patient's course of a treatment regimen involving a CD123×CD3 bispecific binding molecule.

In a second embodiment, a patient is identified as exhibiting an elevated 10-gene expression signature and as thus being particularly amenable to the treatment of hematologic malignancy using the methods and compositions of the present invention by comparing the level of expression of one or more target gene(s) to the averaged or weighted baseline level of expression of such target gene(s) in a population of individuals who are suffering from a hematologic malignancy. A target gene whose expression is greater than such an averaged or weighted baseline level is said to exhibit an “increased” level of expression, and the methods and compositions of the present invention are particularly suitable for use in treating hematologic malignancy in such patients. For example, the methods and compositions of the present invention are particularly suitable for use in patients who exhibit an “increased” level of target gene(s) expression that is greater than the first quartile (i.e., greater than the bottom 25%) of the expression levels of such target gene(s) in a population of individuals who are suffering from a hematologic malignancy. The methods and compositions of the present invention are particularly suitable for use in patients who exhibit an “increased” level of target gene(s) expression that is greater than the second quartile (i.e., greater than the bottom 50%) of the expression levels of such target gene(s) in a population of individuals who are suffering from a hematologic malignancy. The methods and compositions of the present invention are particularly suitable for use in patients who exhibit an “increased” level of target gene(s) expression that is greater than the third quartile (i.e., greater than the bottom 75%) of the expression levels of such target gene(s) in a population of individuals who are suffering from a hematologic malignancy. The methods and compositions of the present invention are particularly suitable for use in patients who exhibit an “increased” level of target gene(s) expression that is greater than 85%, greater than 90%, or greater than 95% of the expression levels of such target gene(s) in a population of individuals who are suffering from a hematologic malignancy.

In a third embodiment, a patient is identified as exhibiting an elevated 10-gene expression signature and as thus being particularly amenable to the treatment of hematologic malignancy using the methods and compositions of the present invention by comparing the level of expression of one or more target gene(s) to the averaged or weighted baseline level of expression of such target gene(s) in a population of individuals who have previously been unsuccessfully treated for a hematologic malignancy using the methods and compositions of the present invention (e.g., a population of individuals who did not successfully respond to a treatment for a hematologic malignancy using a CD123×CD3 bispecific molecule). A target gene whose expression is equal or greater than such an averaged or weighted baseline level is said to exhibit an “increased” level of expression, and the methods and compositions of the present invention are particularly suitable for use in treating hematologic malignancy in such patients. The methods and compositions of the present invention are particularly suitable for use in patients who exhibit an “increased” level of target gene(s) expression that is greater than the first quartile (i.e., greater than the bottom 25%) of the expression levels of such target gene(s) in such population of unsuccessfully-treated individuals. The methods and compositions of the present invention are particularly suitable for use in patients who exhibit an “increased” level of target gene(s) expression that is greater than the second quartile (i.e., greater than the bottom 50%) of the expression levels of such target gene(s) in such population of unsuccessfully-treated individuals. The methods and compositions of the present invention are particularly suitable for use in patients who exhibit an “increased” level of target gene(s) expression that is greater than the third quartile (i.e., greater than the bottom 75%) of the expression levels of such target gene(s) in such population of unsuccessfully-treated individuals. The methods and compositions of the present invention are particularly suitable for use in patients who exhibit an “increased” level of target gene(s) expression that is greater than 85%, greater than 90%, or greater than 95% of the expression levels of such target gene(s) in such population of unsuccessfully-treated individuals.

In a fourth embodiment, a patient is identified as exhibiting an elevated 10-gene expression signature and as thus being particularly amenable to the treatment of hematologic malignancy using the methods and compositions of the present invention by comparing the level of expression of one or more target gene(s) to the averaged or weighted baseline level of expression of such target gene(s) in a population of individuals who have previously been successfully treated for a hematologic malignancy using the methods and compositions of the present invention (e.g., a population of individuals who successfully responded to a treatment for a hematologic malignancy using a CD123×CD3 bispecific molecule). A target gene whose expression is equal or greater than such an averaged or weighted baseline level is said to exhibit an “increased” level of expression, and the methods and compositions of the present invention are particularly suitable for use in treating hematologic malignancy in such patients. The methods and compositions of the present invention are particularly suitable for use in patients who exhibit an “increased” level of target gene(s) expression that is within at least the first quartile (i.e., within the bottom 25%) of the expression levels of such target gene(s) in such population of successfully-treated individuals. The methods and compositions of the present invention are particularly suitable for use in patients who exhibit an “increased” level of target gene(s) expression that is within at least the second quartile (i.e., between the bottom 25% and 50%) of the expression levels of such target gene(s) in such population of successfully-treated individuals. The methods and compositions of the present invention are particularly suitable for use in patients who exhibit an “increased” level of target gene(s) expression that is within at least the third quartile (i.e., between the bottom 50% and 75%) of the expression levels of such target gene(s) in such population of successfully-treated individuals. The methods and compositions of the present invention are even more particularly suitable for use in patients who exhibit an “increased” level of target gene(s) expression that is within at least the fourth quartile (i.e., above the bottom 75%) of the expression levels of such target gene(s) in such population of previously-treated individuals.

In certain embodiments, whether a target gene's expression is “increased” is determined by comparing the level of its expression to the level of expression of one or more genes that are not associated with disease or that do not exhibit increased expression as a consequence of a disease state (“reference” genes). Because reference genes are often expressed at different levels, the geometric mean of the reference genes' expression can be utilized to calculate scaling factors. A geometric mean is obtained by multiplying each gene per sample value in a data set and then taking the n^{th} root (where n is the count of numbers in the set) of the resulting product. A geometric mean is similar to an arithmetic mean, in that it indicates the central tendency of a set of numbers. However, unlike an arithmetic mean, the geometric mean is less sensitive to variation in the magnitude of count levels between probes. To compare biological signatures across a cohort of samples the geometric mean from a set of “reference” gene(s) may be used to normalize individual samples across a data set in order for comparisons between biological genes to be made independent of differences due to technical variation such as sample mass input and sample quality.

Preferred “reference” genes are constitutively expressed at the same level in normal and malignant cells. Housekeeping genes (Eisenberg, E. et al. (2003) “Human Housekeeping Genes Are Compact,” Trends in Genetics. 19(7):362-365; kon Butte, A. J. et al. (2001) “Further Defining Housekeeping, Or “Maintenance,” Genes Focus On ‘A Compendium Of Gene Expression In Normal Human Tissues’,” Physiol. Genomics. 7(2):95-96; Zhu, J. et al. (2008) “On The Nature Of Human Housekeeping Genes,” Trends in Genetics 24(10):481-484; Eisenberg, E. et al. (2013) “Human Housekeeping Genes, Revisited,” Trends in Genetics. 29(10):569-574) such as genes required for the maintenance of basic cellular functions are a preferred class of reference genes.

In a further embodiment, the CD123×CD3 binding molecule therapy of the present invention may additionally comprise the administration of an anti-human PD-L1 binding molecule, such as an anti-human PD-L1 antibody, or a diabody having a human PD-L1 binding domain. Anti-human PD-L1 binding molecules that may be used in accordance with this embodiment include atezolizumab, avelumab, and durvalumab (see, e.g., U.S. Pat. Nos. 9,873,740; 8,779,108). The amino acid sequence of the complete heavy and Light Chains of atezolizumab (WHO Drug Information, 2015, Recommended INN: List 74, 29(3):387), durvalumab (WHO Drug Information, 2015, Recommended INN: List 74, 29(3):393-394) and avelumab (WHO Drug Information, 2016, Recommended INN: List 74, 30(1):100-101) are known in the art.

In an alternative further embodiment, the CD123×CD3 binding molecule therapy of the present invention may additionally comprise the administration of an anti-human PD-1 binding molecule, such as an anti-human PD-1 antibody, or a diabody having a human PD-1 binding domain. Anti-human PD-1 binding molecules that may be used in accordance with this embodiment include: nivolumab (also known as 5C4, BMS-936558, ONO-4538, MDX-1106, and marketed as OPDIVO® by Bristol-Myers Squibb), pembrolizumab (formerly known as lambrolizumab, also known as MK-3475, SCH-900475, and marketed as KEYTRUDA® by Merck), EH12.2H7 (commercially available from BioLegend), pidilizumab (CAS Reg. No.: 1036730-42-3 also known as CT-011, CureTech), retifanlimab (CAS Reg. No.: 2226345-85-1 also know as MGA012), and DART-I (disclosed in WO 2017/019846), (also see, e.g., U.S. Pat. Nos. 5,952,136; 7,488,802; 7,521,051; 8,008,449; 8,088,905; 8,354,509; 8,552,154; 8,779,105; 8,900,587; 9,084,776; PCT Patent Publications WO 2004/056875; WO 2006/121168; WO 2008/156712; WO 2012/135408; WO 2012/145493; WO 2013/014668; WO 2014/179664; WO 2014/194302; WO 2015/112800; WO 2017/019846, and WO 2017/214092).

B. Exemplary “Target” Genes

Table 1 discloses the target genes and a representative, non-limiting GenBank® Accession Number for each gene identified in the 10-gene signature.

TABLE 1

| Gene | GenBank |
|---|---|
| Description | Accession No. † |
| CD8B | NM_004931.5 |
| CRABP2 | NM_001878.4 |
| FCGR3A/B | NM_000569.8/ |
| | NM_001244753.2 |
| FBP1 | NM_000507.4 |
| FPR1 | NM_002029.4 |
| ICOS | NM_012092.4 |
| NOTCH2 | NM_024408.4 |
| PDGFA | NM_002607.5 |
| SERPINH1 | NM_001235.5 |
| THBS1 | NM_003246.4 |

† non-limiting representative example, alleles and/or splice variants are also encompassed.

C. Exemplary “Reference” Genes

Housekeeping genes that are constitutively expressed at the same level in normal and malignant cells comprise a exemplary class of reference genes. Housekeeping genes include genes involved in general gene expression (such as genes encoding transcription factors, repressors, RNA splicing factors, translation factors, tRNA synthetases, RNA binding proteins, ribosomal proteins, mitochondrial ribosomal proteins, RNA polymerases, protein processing factors, heat shock proteins, histones, cell cycle regulators, apoptosis, oncogenes, DNA repair/replication, etc.), metabolism (such as genes encoding enzymes of: carbohydrate metabolism, the citric acid cycle, lipid metabolism, amino acid metabolism, NADH dehydrogenases, cytochrome C oxidase, ATPases, lysosomal enzymes, proteasome proteins, ribonucleases, thioreductases, etc.), cellular structural integrity (such as genes encoding cytoskeletal proteins, proteins involved in organelle synthesis, mitochondrial proteins, etc.), and cell-surface proteins (such as genes encoding cellular adhesion proteins, ion channels and transporters, receptors, HLA/immunoglobulin/cell recognition proteins, etc.), kinases/signaling proteins (such as growth factors, tissue necrosis factor, casein kinase, etc.). Reference genes that are suitable for this purpose include genes that encode:
sterol regulatory element binding proteins (e.g., ATF1, ATF2, ATF4, ATF6, ATF7, ATF7, BTF3, E2F4, ERH, HMGB1, ILF2, IER2, JUND, TCEB2, etc.);
repressors (e.g., PUF60, etc.);
RNA splicing proteins (e.g., BAT1, HNRPD, HNRPK, PABPN1, SRSF3, etc.);
translation factors (e.g., EIF1, EIF1AD, EIFIB, EIF2A, EIF2AK1, EIF2AK3, EIF2AK4, EIF2AK1, EIF2B2, EIF2B3, EIF2B4, EIF2S2, EIF3A, EIF3B, EIF3D, EIF3G, EIF3I, EIF3H, EIF3J, EIF3K, EIF3L, EIF3M, EIF3S5, EIF3S8, EIF4A1, EIF4A2, EIF4A3, EIF4E2, EIF4G1, EIF4G2, EIF4G3, EIF4H, EIF5, EIF5, EIF5A, EIF5AL1, EIF5B, EIF6, TUFM, etc.);
tRNA synthetases (e.g., AARS, AARS2, AARSD1434, CARS, CARS2, DARS, DARS2, EARS2614, FARS2, FARSA, FARSB, GARS, HARS, HARS2, IARS, IARS2, KARS, LARS2, MARS, MARS2, NARS, NARS2, QARS, RARS, RARS2, SARS, TARS, VARS2, WARS2, YARS, YARS2436, etc.);
RNA binding proteins (e.g., ELAVL1, etc.);
ribosomal proteins (e.g., RPL5, RPL8, RPL9, RPL10A, RPL11, RPL14, RPL25, RPL26L1, RPL27, RPL30, RPL32, RPL34, RPL35, RPL35A, RPL36AL, RPS5, RPS6, RPS6KA3, RPS6KB1, RPS6KB2, RPS13, RPS19BP1, RPS20, RPS23, RPS24, RPS27, RPN1, etc.);
mitochondrial ribosomal proteins (e.g., MRPL9, MRPL1, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL35, MRPL36, MRPL37, MRPL38, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS5, MRPS6, MRPS7, MRPS9, etc.);
RNA polymerases (e.g., POLR1C, POLR1D, POLR1E, POLR2A, POLR2B, POLR2C, POLR2D, POLR2E, POLR2F, POLR2G, POLR2H, POLR2I, POLR2J, POLR2K, POLR2L, POLR3C, POLR3E, POLR3GL, POLR3K, etc.);
protein processing proteins (e.g., PPID, PPIE, PPIF, PPIG, PPIH, CANX, CAPN1, CAPN7, CAPNS1, NACA, NACA2, PFDN2, PFDN4, PFDN5, PFDN6, SNX2, SNX3, SNX4, SNX5, SNX6, SNX9, SNX12, SNX13, SNX17, SNX18, SNX19, SNX25, SSR1, SSR2, SSR3, SUMO1, SUMO3, etc.);
heat shock proteins (e.g., HSPA4, HSPA5, HSPA8, HSPA9, HSPA14, HSBP1, etc.);
histones (e.g., HIST1H2BC, H1FX, H2AFV, H2AFX, H2AFY, H2AFZ, etc.);
cell cycle proteins (e.g., ARHGAP35, ARHGAP5, ARHGDIA, ARHGEF10L, ARHGEF11, ARHGEF40, ARHGEF7, RAB10, RAB11A, RAB11B, RAB14, RAB18, RAB1A, RAB1B, RAB21, RAB22A, RAB2A, RAB2B380, RAB3GAP1, RAB3GAP2, RAB40C, RAB4A, RAB5A, RAB5B, RAB5C, RAB6A, RAB7A, RAB9A, RABEPI, RABEPK, RABGEF1, RABGGTA, RABGGTB, CENPB, CTBP1, CCNBIIP1, CCNDBP1, CCNG1, CCNH, CCNK402, CCNL 1, CCNL2, CCNY, PPP1CA, PPP1CC, PPP1R10, PPP1R11, PPP1R15B, PPP1R37, PPP1R7, PPP1R8, PPP2CA, PPP2CB552, PPP2R1A, PPP2R2A, PPP2R2D, PPP2R3C, PPP2R4, PPP2R5A, PPP2R5B, PPP2R5C, PPP2R5D, PPP2R5E, PPP4C, PPP4R1, PPP4R2, PPP5C, PPP6C, PPP6R2, PPP6R3, RAD1, RAD17, RAD23B, RAD50, RAD51C, IST1, etc.);
apoptosis proteins (e.g., DAD1, DAP3, DAXX, etc.);
oncogene proteins (e.g., ARAF, MAZ, MYC, etc.);
DNA repair/replication proteins (e.g., MCM3AP, XRCC5, XRCC6, etc.);
metabolism proteins (e.g., PRKAG1, PRKAA1, PRKAB1, PRKACA, PRKAG1, PRKAR1A, PRKRIP1, etc.);
carbohydrate metabolism proteins (e.g., ALDOA, B3GALT6, B4GALT3, B4GALT5, B4GALT7, GSK3A, GSK3B, TPIl, PGK1, PGAM5, ENOPHI, LDHA, TALDO1, TSTA3);
citric acid cycle proteins (e.g., SDHA, SDHAF2, SDHB, SDHC, SDHD, etc.);
lipid metabolism proteins (e.g., HADHA, etc.);
amino acid metabolism proteins (e.g., COMT, etc.);
NADH dehydrogenases (e.g., NDUFA2, NDUFA3, NDUFA4, NDUFA5, NDUFA6, NDUFA7, NDUFA8, NDUFA9, NDUFA10, NDUFA11, NDUFA12, NDUFA13, NDUFAF2, NDUFAF3, NDUFAF4, NDUFB2, NDUFB3, NDUFB4, NDUFB5, NDUFB6, NDUFB7, NDUFB10, NDUFB11, NDUFB8, NDUFB9, NDUFC1, NDUFC2, NDUFC2, NDUFS5, NDUFV2, NDUFS2, NDUFS3, NDUFS4, NDUFS5, NDUFS6, NDUFS7, NDUFS8, NDUFV1, NDUFV2, etc.);
cytochrome C oxidases (e.g., COX4I1, COX5B, COX6B1, COX6C, COX7A2, COX7A2L, COX7C, COX8, COX8A, COX11, COX14, COX15, COX16, COX19617, COX20, CYC1, UQCC, UQCR10, UQCR11, UQCRB, UQCRC1, UQCRC2, UQCRHL591, UQCRQ, ATPase, ATP2C1, ATP5A1, ATP5B, ATP5C1, ATP5D, ATP5F1, ATP5G2, ATP5G3, ATP5H, ATP5J, ATP5J2, ATP5J2, ATP5L, ATP50, ATP5S, ATP5SL, ATP6AP1, ATP6VOA2, ATP6VOB, ATP6VOC, ATP6VOD1, ATP6VOE1, ATP6V1C1, ATP6V1D, ATP6V1E1, ATP6V1F, ATP6V1G1, ATP6V1H, ATPAF2, ATPIF1, etc.);
lysosomal proteins (e.g., CTSD, CSTB, LAMP1, LAMP2, M6PR, etc.);
proteasomal proteins (e.g., PSMA1, PSMA2, PSMA3, PSMA4, PSMA5, PSMA6, PSMA7, PSMB1, PSMB2, PSMB3, PSMB4, PSMB5, PSMB6, PSMB7, PSMC2, PSMC3, PSMC4, PSMC5, PSMC6, PSMD1, PSMD10, PSMD11, PSMD12, PSMD13, PSMD14, PSMD2, PSMD3, PSMD4, PSMD5, PSMD6, PSMD7, PSMD8, PSMD9, PSME2, PSME3, PSMF1, PSMG2, PSMG3, PSMG4591, UBA1, UBA2, UBA3, UBA5, UBA52, UBAC2, UBALDI, UBAP1, UBAP2L, UBB, UBC, UBE2A, UBE2B, UBE2D2, UBE2D3, UBE2D4, UBE2E1, UBE2E2, UBE2E3, UBE2F, UBE2G2, UBE2H, UBE2I, UBE2J1, UBE2J2, UBE2K, UBE2L3, UBE2M, UBE2N, UBE2NL989, UBE2Q1, UBE2R2, UBE2V1, UBE2V2, UBE2W, UBE2Z, UBE3A, UBE3B, UBE3C, UBE4A, UBE4B, USP10, USP14, USP16, USP19, USP22, USP25, USP27X073, USP33, USP38, USP39, USP4, USP47, USP5, USP7, USP8, USP9X590, etc.);
ribonucleases (e.g., RNH, etc.);
thioreductases (e.g., TXN2, TXNDC11, TXNDC12, TXNDC15, TXNDC17, TXNDC9, TXNL1, TXNL4A, TXNL4B, TXNRD1, Cytoskeletal, ANXA6, ANXA7, ARPC1A, ARPC2, ARPC5L, CAPZA2, CAPZB, RHOB, RHOT1, RHOT2, TUBB, WDR1, etc.);
proteins involved in organelle synthesis (e.g., BLOC1S1, BLOC1S2, BLOC1S3, BLOC1S4, BLOC1S6, AP1G1, AP1M1, AP2A1, AP2A2, AP2M1, AP2S1, AP3B1, AP3D1, AP3M1, AP3S1, AP3S2, AP4B1, AP5M1, ANXA6, ANXA7, AP1B1, CLTA, CLTB, CLTC, etc.);
mitochondrial proteins (e.g., MTX2, etc.);
cell surface proteins (e.g., AP2S1, CD81, GPAA1, LGALS9, MGAT2, MGAT4B, VAMP3, etc.);
cell adhesion proteins (e.g., CTNNA1, CTNNB1, CTNNBIP1, CTNNBL1, CTNND1458, etc.);
ion channels and transporter proteins (e.g., ABCB10, ABCB7, ABCD3, ABCE1, ABCF1, ABCF2, ABCF3, CALM1, MFSD11, MFSD12, MFSD3, MFSD5, SLC15A4, SLC20A1, SLC25A11, SLC25A26, SLC25A28, SLC25A3, SLC25A32, SLC25A38, SLC25A39, SLC25A44, SLC25A46, SLC25A5, SLC27A4, SLC30A1, SLC30A5, SLC30A9, SLC35A2, SLC35A4, SLC35B1, SLC35B2, SLC35C2, SLC35E1, SLC35E3, SLC35F5, SLC38A2, SLC39A1, SLC39A3, SLC39A7, SLC41A3, SLC46A3, SLC48A1, Receptors, ACVR1, ACVR1B, CD23, etc.);
HLA/immunoglobulin/cell recognition proteins (e.g., BAT1, BSG, MIF, TAPBP, etc.);
kinases/signaling proteins (e.g., ADRBK1, AGPAT1, ARF1, ARF3, ARF4, ARF5, ARL2, CSF1, CSK, DCT, EFNA3, FKBPlA, GDI1, GNAS1, GNAI2, HAX1, ILK, MAPKAPK2, MAP2K2, MAP3K11, PITPNM, RAC1, RAP1B, RAGA, STK19, STK24, STK25, YWHAB, YWHAH, YWHAQ, YWHAZ, etc.);
growth factors (e.g., AIF1, HDGF, HGS, LTBP4, VEGFB, ZFP36L1, tissue necrosis factor, CD40, casein kinase, CSNK1E, CSNK2B, etc.); and
miscellaneous proteins (e.g., ALAS1, ARHGEF2, ARMET, AES, BECN1, BUD31, CKB, CPNE1, ENSA, FTH1, GDI2, GUK1, HPRT, IFITMI, JTB, MMPL2, NME2, NONO, P4HB, PRDX1, PTMA, RPA2, SULT1A3, SYNGR2, TTC1, C11Orfl3, C14orf2, C21orf33, SPAG7, SRM, TEGT, DAZAP2, MEA1, etc.).

Exemplary housekeeping genes include those listed in Table 2. Table 2 also provides a representative, non-limiting NCBI Accession Number for each gene. Any combination or sub-combination of such genes (and/or splice variants of the same) may be employed.

TABLE 2

| | NCBI | | |
|---|---|---|---|
| Official | Accession | Gene | Full Name |
| Symbol | No. | ID | |
| ABCF1 | NM_ | 23 | Homo sapiens ATP-binding cassette, |
| | 001090.2 | | sub-family F (GCN20), member 1 |
| | | | (ABCF1), transcript variant 2, |
| | | | mRNA |
| ACTB | NM_ | 60 | Homo sapiens actin, beta (ACTB), |
| | 001101.2 | | mRNA |
| ALAS1 | NM_ | 211 | Homo sapiens aminolevulinate, |
| | 000688.4 | | delta-, synthase 1 (ALAS1), |
| | | | transcript variant 1, mRNA |
| B2M | NM_ | 567 | Homo sapiens beta-2-microglobulin |
| | 004048.2 | | (B2M), mRNA |
| CLTC | NM_ | 1213 | Homo sapiens clathrin, heavy |
| | 004859.2 | | polypeptide (Hc) (CLTC), mRNA |
| EEF1G | NM_ | 1937 | Homo sapiens eukaryotic translation |
| | 001404.4 | | elongation factor 1 gamma |
| | | | (EEF1G), mRNA |
| G6PD | NM_ | 2539 | Homo sapiens glucose-6-phosphate |
| | 000402.2 | | dehydrogenase (G6PD), nuclear |
| | | | gene encoding mitochondrial |
| | | | protein, mRNA |
| GAPDH | NM_ | 2597 | Homo sapiens glyceraldehyde-3- |
| | 002046.3 | | phosphate dehydrogenase |
| | | | (GAPDH), mRNA |
| GUSB | NM_ | 2990 | Homo sapiens glucuronidase, beta |
| | 000181.1 | | (GUSB), mRNA |
| HPRT1 | NM_ | 3251 | Homo sapiens hypoxanthine |
| | 000194.1 | | phosphoribosyltransferase 1 (Lesch- |
| | | | Nyhan syndrome) (HPRT1), mRNA |
| LDHA | NM_ | 3939 | Homo sapiens lactate |
| | 005566.1 | | dehydrogenase A (LDHA), mRNA |
| NRDE2 | NM_ | 55051 | Necessary For RNA Interference, |
| | 017970.3 | | Domain Containing |
| OAZ1 | NM_ | 4946 | Homo sapiens ornithine |
| | 004152.3 | | decarboxylase antizyme 1 (OAZ1), |
| | | | transcript variant 1, mRNA |
| PGK1 | NM_ | 5230 | Homo sapiens phosphoglycerate |
| | 000291.2 | | kinase 1 (PGK1), mRNA |
| POLR1B | NM_ | 84172 | Homo sapiens polymerase (RNA) I |
| | 019014.3 | | polypeptide B, 128 kDa (POLR1B), |
| | | | mRNA |
| POLR2A | NM_ | 5430 | Homo sapiens polymerase (RNA) II |
| | 000937.2 | | (DNA directed) polypeptide A, 220 |
| | | | kDa (POLR2A), mRNA |
| PPIA | NM_ | 5478 | Homo sapiens peptidylprolyl |
| | 021130.4 | | isomerase A (PPIA), transcript |
| | | | variant 1, mRNA |
| RPL19 | NM_ | 6143 | Homo sapiens ribosomal protein |
| | 000981.3 | | L19 (RPL19), mRNA |
| RPLP0 | NM_ | 6175 | Homo sapiens ribosomal protein, |
| | 001002.3 | | large, P0 (RPLP0), transcript |
| | | | variant 1, mRNA |
| SDHA | NM_ | 6389 | Homo sapiens succinate |
| | 004168.1 | | dehydrogenase complex, subunit |
| | | | A, flavoprotein (Fp) (SDHA), |
| | | | nuclear gene encoding |
| | | | mitochondrial protein, mRNA |
| STK11IP | NM_ | 114790 | Serine/Threonine Kinase 11 |
| | 052902.3 | | Interacting Protein |
| TBC1D10B | NM_ | 26000 | TBC1 domain family, member 10B |
| | 015527.3 | | |
| TBP | NM_ | 6908 | Homo sapiens TATA-box binding |
| | 003194.3 | | protein (TBP), transcript variant 1, |
| | | | mRNA |
| TBP | NM_ | 6908 | Homo sapiens TATA-box binding |
| | 001172085.1 | | protein (TBP), transcript variant 2, |
| | | | mRNA |
| TUBB | NM_ | 203068 | Homo sapiens tubulin, beta |
| | 178014.2 | | (TUBB), mRNA |
| UBB | NM_ | 7314 | Ubiquitin B |
| | 018955.3 | | |

In certain embodiments, the following reference genes are utilized ABCF1, G6PD, NRDE2, OAZ1, POLR2A, SDHA, STK11IP, TBC1D10B, TBP, and UBB).

D. Exemplary Methods for Evaluating Expression of Target and Reference Genes

In order to reveal the level of expression of the target gene(s) relative to the baseline or reference gene(s), the amount of mRNA in a cellular sample corresponding to each assessed target gene is determined and normalized to the expression of mRNA corresponding to the baseline or reference gene(s). Any suitable method may be employed to accomplish such an analysis. A exemplary method employs the nCOUNTER® Analysis System (NanoString Technologies, Inc.). In the nCOUNTER® Analysis System, RNA of a sample is incubated in the presence of sets of gene-specific Reporter Probes and Capture Probes under conditions sufficient to permit the sample RNA to hybridize to the probes. Each Reporter Probe carries a fluorescent barcode and each Capture Probe contains a biotin moiety capable of immobilizing the hybridized complex to a solid support for data collection. After hybridization, excess probe is removed, and the support is scanned by an automated fluorescence microscope. Barcodes are counted for each target molecule. Data analysis is may be conducted using nSolver® 4.0 Analysis Software (NanoString Technologies, Inc.), or similar. The data presented in Example 1 was obtained using PanCancer IO 360™ Gene Expression Panel kits (NanoString Technologies, Inc.) which contain a set of probes for 770 different genes (750 genes cover the key pathways at the interface of the tumor, tumor microenvironment, and immune response, and 20 internal reference genes that may be used for data normalization (Table 5). The 10-gene signature score is calculated as follows:
Raw data counts for each gene are normalized to the geometric mean of the selected housekeeping (HK) genes (e.g., ABCF1, NRDE2, G6PD, OAZ1, POLR2A, SDHA, STK11IP, TBC1D10B, TBP, UBB) for each sample.
HK normalized data is then normalized to IO 360 panel standards, preferably to those run on the same cartridges as the test samples.
Each normalized gene count is then log transformed.
Each of the normalized and log transformed RNA values is summed together.
The sum of the normalized log transformed RNA values is divided by the number of target genes in the signature (i.e., 10) to generate a single score.

II. Exemplary CD123×CD3 Bispecific Binding Molecules

A. JNJ-63709178

JNJ-63709178 is a humanized IgG4 bispecific antibody with silenced Fc function. The antibody was produced using Genmab DuoBody® technology and is able to bind both CD123 on tumor cells and CD3 on T cells. JNJ-63709178 is able to recruit T cells to CD123-expressing tumor cells and induce the killing of these tumor cells in vitro (MOLM-13, OCI-AML5 and KG-1; EC50=0.51−0.91 nM). JNJ-63709178 is disclosed in WO 2016/036937, Gaudet, F. et al. (2016) “Development of a CD123×CD3 Bispecific Antibody (JNJ-63709178) for the Treatment of Acute Myeloid Leukemia (AML),” Blood 128:2824; and Forslund, A. et al. (2016) “Ex Vivo Activity Profile of the CD123×CD3 Duobody® Antibody JNJ-63709178 Against Primary Acute Myeloid Leukemia Bone Marrow Samples,” Blood 128:2875, which documents are herein incorporated by reference). The amino acid sequences of the heavy and light chains of JNJ-63709178 and/or related antibodies: 13RB179, 13RB180, 13RB181, 13RB182, 13RB183, 13RB186, 13RB187, 13RB188, 13RB189, CD3B19, 7959, 3978, 7955, 9958, 8747, 8876, 4435 and 5466 are disclosed in WO 2016/036937.

B. XmAb14045

XmAb14045 (also known as vibecotamab) is a tumor-targeted antibody that contains both a CD123 binding domain and a cytotoxic T-cell binding domain (CD3). An XmAb Bispecific Fc domain serves as the scaffold for these two antigen binding domains and confers long circulating half-life, stability and ease of manufacture on XmAb14045. Engagement of CD3 by XmAb14045 activates T cells for highly potent and targeted killing of CD123-expressing tumor cells (US Patent Publication 2017/0349660; Chu, S. Y. et al. (2014) “Immunotherapy with Long-Lived Anti-CD123× CD3 Bispecific Antibodies Stimulates Potent T Cell-Mediated Killing of Human AML Cell Lines and of CD123+ Cells in Monkeys: A Potential Therapy for Acute Myelogenous Leukemia,” Blood 124(21):2316, which documents are herein incorporated by reference). The amino acid sequences of the heavy and light chains of XmAb14045 and similar CD123×CD3 bispecific binding molecules are disclosed in US Patent Publication 2017/0349660 and in WHO Drug Information, Proposed INN: List 120, 2018, 32(4):658-660.

C. APVO436

APVO436 is an ADAPTIR™ CD123×CD3 bispecific binding molecule that possesses an anti-CD123 scFv portion and an anti-CD3 scFv portion. Each of the scFv portions are bound to an Fc Domain that has been modified to abolish ADCC/CDC effector function. APVO436 is disclosed to bind human CD123 and CD3-expressing cells with EC₅₀ values in the low nM range and to demonstrate potent target-specific activity against CD123-expressing tumor cell lines at low effector to target ratios. APVO436 is disclosed to be capable of potently inducing endogenous T-cell activation and proliferation accompanied by depletion of CD123 expressing cells in experiments with primary AML subject samples and normal donor samples. APVO436 (see, Comeau, M. R. et al. (2018) “APVO436, a Bispecific anti-CD123×anti-CD3 ADAPTIR™ Molecule for Redirected T-cell Cytotoxicity, Induces Potent T-cell Activation, Proliferation and Cytotoxicity with Limited Cytokine Release,” AACR Annual Meeting April 2018, Abstract 1786; Godwin, C. D. et al. (2017) “Bispecific Anti-CD123×Anti-CD3 ADAPTIR™ Molecules APVO436 andAPVO437Have Broad Activity Against Primary Human AML Cells In Vitro,” American Society of Hematology Annual Meeting, December 2017, Blood 130:2639; Comeau, M. R. et al. (2017) “Bispecific anti-CD123×anti-CD3 ADAPTIR™ Molecules for Redirected T-cell Cytotoxicity in Hematological Malignancies,” AACR Annual Meeting April 2017, Abstract 597). The amino acid sequences of the heavy and light chains of APVO436 CD123×CD3 bispecific binding molecules are disclosed in WO 2018/057802A1.

D. DART-A

DART-A (also known as flotetuzumab, CAS number: 1664355-28-5) is an exemplary CD123×CD3 bispecific binding molecule of the present invention. DART-A is a sequence-optimized bispecific diabody capable of simultaneously and specifically binding to an epitope of CD123 and to an epitope of CD3 (a “CD123×CD3” bispecific diabody) (US Patent Publn. No. US 2016-0200827, in PCT Publn. WO 2015/026892, in Al-Hussaini, M. et al. (2016) “Targeting CD123 In Acute Myeloid Leukemia Using A T-Cell-Directed Dual-Affinity Retargeting Platform,” Blood 127:122-131, in Vey, N. et al. (2017) “A Phase I, First-in-Human Study of MGDO06 S80880 (CD123×CD3) in AML MDS,” 2017 ASCO Annual Meeting, Jun. 2-6, 2017, Chicago, TL: Abstract TPS7070, each of which documents is herein incorporated by reference in its entirety). DART-A was found to exhibit enhanced functional activity relative to other non-sequence-optimized CD123×CD3 bispecific diabodies of similar composition, and is thus termed a “sequence-optimized” CD123×CD3 bispecific diabody. PCT Application PCT/US2017/050471 describes particular dosing regimens for administering DART-A to patients, and is herein incorporated by reference in its entirety.

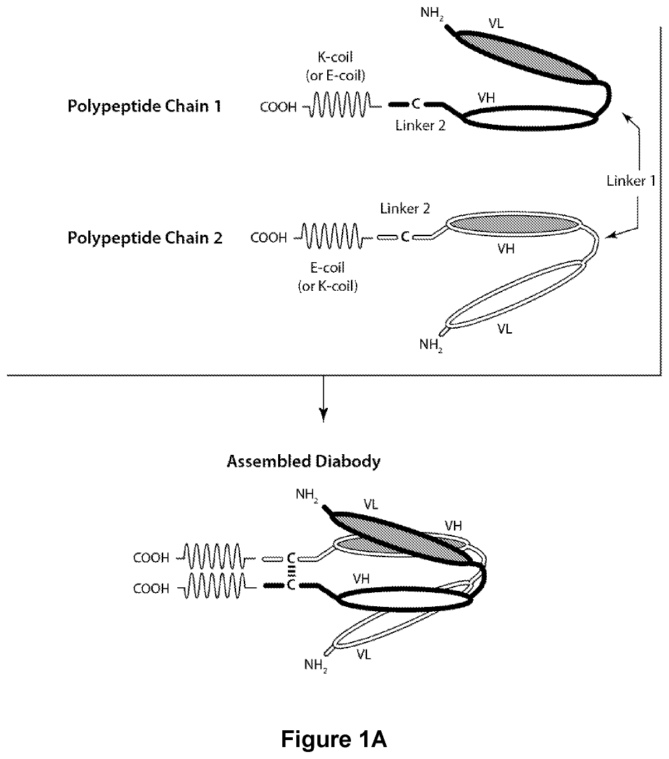

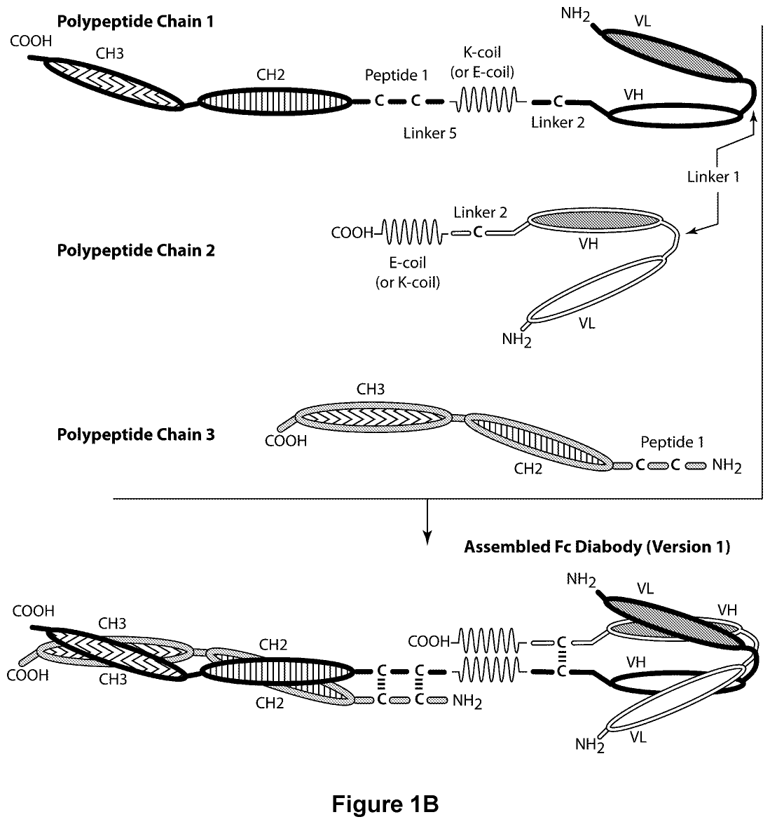

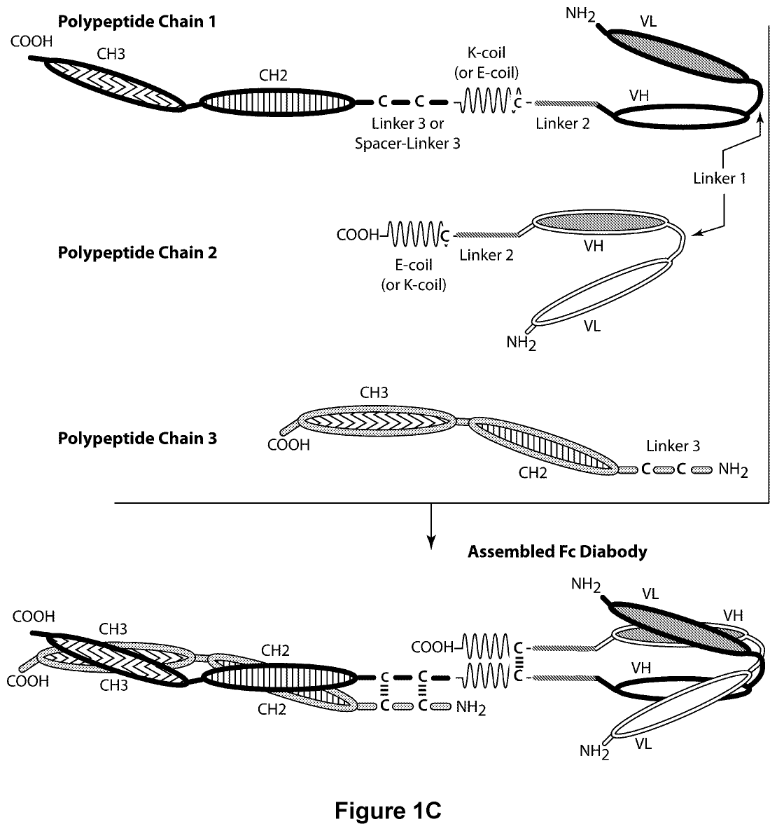

DART-A comprises a first polypeptide chain and a second polypeptide chain (FIG. 1). The first polypeptide chain of the bispecific diabody will comprise, in the N-terminal to C-terminal direction, an N-terminus, a Light Chain Variable Domain (VL Domain) of a monoclonal antibody capable of binding to CD3 (VL_{CD3}), an intervening linker peptide (Linker 1), a Heavy Chain Variable Domain (VH Domain) of a monoclonal antibody capable of binding to CD123 (VH_{CD123}), and a C-terminus.

An exemplary sequence for such a VL_{CD3} Domain is

SEQ ID NO:1:
QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG

The Antigen Binding Domain of VL_{CD3} comprises:

CDR_{L}1 (SEQ ID NO: 2):
RSSTGAVTTSNYAN
CDR_{L}2 (SEQ ID NO: 3):
GTNKRAP
CDR_{L}3 (SEQ ID NO: 4):
ALWYSNLWV

An exemplary sequence for such Linker 1 is

SEQ ID NO: 5:
GGGSGGGG.

An exemplary sequence for such a VH_{CD123} Domain is

SEQ ID NO: 6:
EVQLVQSGAE LKKPGASVKV SCKASGYTFT DYYMKWVRQA
PGQGLEWIGD IIPSNGATFY NQKFKGRVTI TVDKSTSTAY
MELSSLRSED TAVYYCARSH LLRASWFAYW GQGTLVTVSS

The Antigen Binding Domain of VH_{CD123} comprises:

| | |
|---|---|
| CDR_{H}1 (SEQ ID NO: 7): | DYYMK |
| CDR_{H}2 (SEQ ID NO: 8): | DIIPSNGATFYNQKFKG |
| CDR_{H}3 (SEQ ID NO: 9): | SHLLRASWFAY |

The second polypeptide chain will comprise, in the N-terminal to C-terminal direction, an N-terminus, a VL domain of a monoclonal antibody capable of binding to CD123 (VL_{CD123}), an intervening linker peptide (e.g., Linker 1), a VH domain of a monoclonal antibody capable of binding to CD3 (VH_{CD3}), and a C-terminus. An exemplary sequence for such a VL_{CD123} Domain is

SEQ ID NO: 10:
DEVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIK

The Antigen Binding Domain of VL_{CD123} comprises:

| | |
|---|---|
| CDR_{L}1 (SEQ ID NO: 11): | KSSQSLLNSGNQKNYLT |
| CDR_{L}2 (SEQ ID NO: 12): | WASTRES |
| CDR_{L}3 (SEQ ID NO: 13): | QNDYSYPYT |

An exemplary sequence for such a VH_{CD3} Domain is

SEQ ID NO: 14:
EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL
VTVSS

The Antigen Binding Domain of VH_{CD3} comprises:

| | |
|---|---|
| CDR_{H}1 (SEQ ID NO: 15): | TYAMN |
| CDR_{H}2 (SEQ ID NO: 16): | RIRSKYNNYATYYADSVKD |
| CDR_{H}3 (SEQ ID NO: 17): | HGNFGNSYVSWFAY |

The sequence-optimized CD123×CD3 bispecific diabodies of the present invention are engineered so that such first and second polypeptides covalently bond to one another via cysteine residues along their length. Such cysteine residues may be introduced into the intervening linker (e.g., Linker 1) that separates the VL and VH domains of the polypeptides. Alternatively, a second peptide (Linker 2) is introduced into each polypeptide chain, for example, at a position N-terminal to the VL domain or C-terminal to the VH domain of such polypeptide chain. An exemplary sequence for such Linker 2 is SEQ ID NO:18: GGCGGG.

The formation of heterodimers can be driven by further engineering such polypeptide chains to contain polypeptide coils of opposing charge. Thus, in a particular embodiment, one of the polypeptide chains will be engineered to contain an “E-coil” domain (SEQ ID NO:19: EVAALEKEVAALEKEVAALEKEVAALEK) whose residues will form a negative charge at pH 7, while the other of the two polypeptide chains will be engineered to contain an “K-coil” domain (SEQ ID NO:20: KVAALKEKVAALKEKVAALKEKVAALKE) whose residues will form a positive charge at pH 7. The presence of such charged domains promotes association between the first and second polypeptides, and thus fosters heterodimerization.

It is immaterial which coil is provided to the first or second polypeptide chains. However, an exemplary sequence-optimized CD123×CD3 bispecific diabody of the present invention (“DART-A”) has a first polypeptide chain having the sequence

(SEQ ID NO: 21):
QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVQSGAELK KPGASVKVSC KASGYTETDY YMKWVRQAPG
QGLEWIGDII PSNGATFYNQ KFKGRVTITV DKSTSTAYME
LSSLRSEDTA VYYCARSHLL RASWFAYWGQ GTLVTVSSGG
CGGGEVAALE KEVAALEKEV AALEKEVAAL EK

DART-A Chain 1 is composed of: SEQ ID NO:1-SEQ ID NO:5-SEQ ID NO:6-SEQ ID NO:18-SEQ ID NO:19. A polynucleotide that encodes the first polypeptide chain of DART-A is

SEQ ID NO: 22:
caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggcgg aggcgaggtg
cagctggtgc agtccggggc tgagctgaag aaacccggag
cttccgtgaa ggtgtcttgc aaagccagtg gctacacctt
cacagactac tatatgaagt gggtcaggca ggctccagga
cagggactgg aatggatcgg cgatatcatt ccttccaacg
gggccacttt ctacaatcag aagtttaaag gcagggtgac
tattaccgtg gacaaatcaa caagcactgc ttatatggag
ctgagctccc tgcgctctga agatacagcc gtgtactatt
gtgctcggtc acacctgctg agagccagct ggtttgctta
ttggggacag ggcaccctgg tgacagtgtc ttccggagga
tgtggcggtg gagaagtggc cgcactggag aaagaggttg
ctgctttgga gaaggaggtc gctgcacttg aaaaggaggt
cgcagccctg gagaaa

The second polypeptide chain of DART-A has the sequence

(SEQ ID NO: 23):
DFVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIKGGGSGGG
GEVQLVESGG GLVQPGGSLR LSCAASGFTF STYAMNWVRQ
APGKGLEWVG RIRSKYNNYA TYYADSVKDR FTISRDDSKN
SLYLQMNSLK TEDTAVYYCV RHGNFGNSYV SWFAYWGQGT
LVTVSSGGCG GGKVAALKEK VAALKEKVAA LKEKVAALKE

DART-A Chain 2 is composed of: SEQ ID NO:10-SEQ ID NO:5-SEQ ID NO:14-SEQ ID NO:18-SEQ ID NO:20. A polynucleotide that encodes the second polypeptide chain of DART-A is

SEQ ID NO: 24
gacttcgtga tgacacagtc tcctgatagt ctggccgtga
gtctggggga gcgggtgact atgtcttgca agagctccca
gtcactgctg aacagcggaa atcagaaaaa ctatctgacc
tggtaccagc agaagccagg ccagccccct aaactgctga
tctattgggc ttccaccagg gaatctggcg tgcccgacag
attcagcggc agcggcagcg gcacagattt taccctgaca
atttctagtc tgcaggccga ggacgtggct gtgtactatt
gtcagaatga ttacagctat ccctacactt tcggccaggg
gaccaagctg gaaattaaag gaggcggatc cggcggcgga
ggcgaggtgc agctggtgga gtctggggga ggcttggtcc
agcctggagg gtccctgaga ctctcctgtg cagcctctgg
attcaccttc agcacatacg ctatgaattg ggtccgccag
gctccaggga aggggctgga gtgggttgga aggatcaggt
ccaagtacaa caattatgca acctactatg ccgactctgt
gaaggataga ttcaccatct caagagatga ttcaaagaac
tcactgtatc tgcaaatgaa cagcctgaaa accgaggaca
cggccgtgta ttactgtgtg agacacggta acttcggcaa
ttcttacgtg tcttggtttg cttattgggg acaggggaca
ctggtgactg tgtcttccgg aggatgtggc ggtggaaaag
tggccgcact gaaggagaaa gttgctgctt tgaaagagaa
ggtcgccgca cttaaggaaa aggtcgcagc cctgaaagag

DART-A has the ability to simultaneously bind CD123 and CD3 as arrayed by human and cynomolgus monkey cells. Provision of DART-A was found to cause T cell activation, to mediate blast reduction, to drive T cell expansion, to induce T cell activation and to cause the redirected killing of target cancer cells (Table 3).

TABLE 3
Equilibrium Dissociation Constants (K_{D}) for the Binding of DART-A
to Human and Cynomolgus Monkey CD3 and CD123

| | kₐ (±SD) | k_{d} (±SD) | K_{D} (±SD) |
|---|---|---|---|
| Antigens | (M⁻¹s⁻¹) | (s⁻¹) | (nM) |
| Human CD3ε/δ | 5.7 (±0.6) × 10⁵ | 5.0 (±0.9) × 10⁻³ | 9.0 ± 2.3 |
| Cynomolgus CD3ε/δ | 5.5 (±0.5) × 10⁵ | 5.0 (±0.9) × 10⁻³ | 9.2 ± 2.3 |
| Human CD123-His | 1.6 (±0.4) × 10⁶ | 1.9 (±0.4) × 10⁻⁴ | 0.13 ± 0.01 |
| Cynomolgus CD123-His | 1.5 (±0.3) × 10⁶ | 4.0 (±0.7) × 10⁻⁴ | 0.27 ± 0.02 |

More particularly, DART-A exhibits a potent redirected killing ability with concentrations required to achieve 50% of maximal activity (EC50s) in sub-ng/mL range, regardless of CD3 epitope binding specificity in target cell lines with high CD123 expression (Kasumi-3 (EC₅₀=0.01 ng/mL)) medium CD123-expression (Molm13 (EC₅₀=0.18 ng/mL) and THP-1 (EC₅₀=0.24 ng/mL)) and medium low or low CD123 expression (TF-1 (EC₅₀=0.46 ng/mL) and RS4-11 (EC₅₀=0.5 ng/mL)). Similarly, DART-A-redirected killing was also observed with multiple target cell lines with T cells from different donors and no redirected killing activity was observed in cell lines that do not express CD123. Results are summarized in Table 4.

TABLE 4

| | | EC₅₀ of Sequence- | |
|---|---|---|---|
| | CD123 Surface | Optimized CD 123 × | |
| | Expression | CD3 Bispecific | |
| Target Cell | (Antibody | Diabodies (ng/mL) | |
| Line | Binding Sites) | E:T = 10:1 | Max % Killing |
| Kasumi-3 | 118620 | 0.01 | 94 |
| Molm13 | 27311 | 0.18 | 43 |
| THP-1 | 58316 | 0.24 | 40 |
| TF-1 | 14163 | 0.46 | 46 |
| RS4-11 | 957 | 0.5 | 60 |
| A498 | Negative | No activity | No activity |
| HT29 | Negative | No activity | No activity |

Additionally, when human T cells and tumor cells (Molm13 or RS4-11) were combined and injected subcutaneously into NOD/SCID gamma (NSG) knockout mice, the MOLM13 tumors was significantly inhibited at the 0.16, 0.5, 0.2, 0.1, 0.02, and 0.004 mg/kg dose levels. A dose of 0.004 mg/kg and higher was active in the MOLM13 model. The lower DART-A doses associated with the inhibition of tumor growth in the MOLM13 model compared with the RS4-11 model are consistent with the in vitro data demonstrating that MOLM13 cells have a higher level of CD123 expression than RS4-11 cells, which correlated with increased sensitivity to DART-A-mediated cytotoxicity in vitro in MOLM13 cells.

DART-A is active against primary AML specimens (bone marrow mononucleocytes (BMNC) and peripheral blood mononucleocytes (PBMC)) from AML patients. Incubation of primary AML bone marrow samples with DART-A resulted in depletion of the leukemic cell population over time, accompanied by a concomitant expansion of the residual T cells (both CD4 and CD8) and the induction of T cell activation markers (CD25 and Ki-67). Upregulation of granzyme B and perforin levels in both CD8 and CD4 T cells was observed. Incubation of primary AML bone marrow samples with DART-A resulted in depletion of the leukemic cell population over time compared to untreated control or Control DART. When the T cells were counted (CD8 and CD4 staining) and activation (CD25 staining) were assayed, the T cells expanded and were activated in the DART-A sample compared to untreated or Control DART samples. DART-A was also found to be capable of mediating the depletion of pDCs cells in both human and cynomolgus monkey PBMCs, with cynomolgus monkey pDCs being depleted as early as 4 days post infusion with as little as 10 ng/kg DART-A. No elevation in the levels of cytokines interferon gamma, TNF alpha, IL6, IL5, IL4 and IL2 were observed in DART-A-treated animals. These data indicate that DART-A-mediated target cell killing was mediated through a granzyme B and perforin pathway.

No activity was observed against CD123-negative targets (U937 cells) or with Control DART, indicating that the observed T cell activation was strictly dependent upon target cell engagement and that monovalent engagement of CD3 by DART-A was insufficient to trigger T cell activation.

In sum, DART-A is an antibody-based molecule engaging the CD3F subunit of the TCR to redirect T lymphocytes against cells expressing CD123, an antigen up-regulated in several hematologic malignancies. DART-A binds to both human and cynomolgus monkey's antigens with similar affinities and redirects T cells from both species to kill CD123+ cells. Monkeys infused 4 or 7 days a week with weekly escalating doses of DART-A showed depletion of circulating CD123+ cells 72 h after treatment initiation that persisted throughout the 4 weeks of treatment, irrespective of dosing schedules. A decrease in circulating T cells also occurred, but recovered to baseline before the subsequent infusion in monkeys on the 4-day dose schedule, consistent with DART-A-mediated mobilization. DART-A administration increased circulating PD1+, but not TIM-3+, T cells; furthermore, ex vivo analysis of T cells from treated monkeys exhibited unaltered redirected target cell lysis, indicating no exhaustion. Toxicity was limited to a minimal transient release of cytokines following the DART-A first infusion, but not after subsequent administrations even when the dose was escalated, and a minimal reversible decrease in red cell mass with concomitant reduction in CD123+bone marrow progenitors.

E. Additional Bispecific Diabody Molecules

An alternative version of DART-A comprising an Fe Region and having the general structure shown in FIG. 1B is described in US 2016-0200827. Exemplary polypeptides that contains the CH2 and CH3 Domains of an Fc Domain have the sequence

(SEQ ID NO: 25) (“Knob-Bearing” Fc Domain)
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGX

wherein X is K or is absent and the sequence

(SEQ ID NO: 26) (“Hole-Bearing” Fc Domain)
APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGX

wherein X is K or is absent

The first polypeptide of an exemplary DART-A w/Fc construct comprises, in the N-terminal to C-terminal direction, an N-terminus, a VL domain of a monoclonal antibody capable of binding to CD123 (VL_{CD123}), an intervening linker peptide (Linker 1), a VH domain of a monoclonal antibody capable of binding to CD3 (VH_{CD3}), a Linker 2, an E-coil Domain, a Linker 5, Peptide 1, a polypeptide that contains the CH2 and CH3 Domains of an Fc Domain and a C-terminus. An exemplary Linker 5 has the sequence: GGG. An exemplary Peptide 1 has the sequence: DKTHTCPPCP (SEQ ID NO:29). Thus, the first polypeptide of such a DART-A w/Fc version 1 construct is composed of: SEQ ID NO:10-SEQ ID NO:5-SEQ ID NO:14-SEQ ID NO:18 SEQ ID NO:19-GGG-SEQ ID NO:29-SEQ ID NO:25 (wherein X is K).

An exemplary sequence of the first polypeptide of such a DART-A w/Fc version 1 construct has the sequence

(SEQ ID NO: 27):
DEVMTQSPDS LAVSLGERVT MSCKSSQSLL NSGNQKNYLT
WYQQKPGQPP KLLIYWASTR ESGVPDRFSG SGSGTDFTLT
ISSLQAEDVA VYYCQNDYSY PYTFGQGTKL EIKGGGSGGG
GEVQLVESGG GLVQPGGSLR LSCAASGFTF STYAMNWVRQ
APGKGLEWVG RIRSKYNNYA TYYADSVKDR FTISRDDSKN
SLYLQMNSLK TEDTAVYYCV RHGNFGNSYV SWFAYWGQGT
LVTVSSGGCG GGEVAALEKE VAALEKEVAA LEKEVAALEK
GGGDKTHTCP PCPAPEAAGG PSVFLFPPKP KDTLMISRTP
EVTCVVVDVS HEDPEVKFNW YVDGVEVHNA KTKPREEQYN
STYRVVSVLT VLHQDWLNGK EYKCKVSNKA LPAPIEKTIS
KAKGQPREPQ VYTLPPSREE MTKNQVSLWC LVKGEYPSDI
AVEWESNGQP ENNYKTTPPV LDSDGSFFLY SKLTVDKSRW
QQGNVFSCSV MHEALHNHYT QKSLSLSPGK

The second chain of such a DART-A w/Fc version 1 construct will comprise, in the N-terminal to C-terminal direction, an N-terminus, a VL domain of a monoclonal antibody capable of binding to CD3 (VL_{CD3}), an intervening linker peptide (Linker 1), a VH domain of a monoclonal antibody capable of binding to CD123 (VH_{CD123}), a Linker 2, a K-coil Domain, and a C-terminus. Thus, the second polypeptide of such a DART-A w/Fc version 1 construct is composed of: SEQ ID NO:1-SEQ ID NO:5-SEQ ID NO:6-SEQ ID NO:18-SEQ ID NO:20. Such a polypeptide has the sequence

(SEQ ID NO: 28):
QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVQSGAELK KPGASVKVSC KASGYTETDY YMKWVRQAPG
QGLEWIGDII PSNGATFYNQ KFKGRVTITV DKSTSTAYME
LSSLRSEDTA VYYCARSHLL RASWFAYWGQ GTLVTVSSGG
CGGGKVAALK EKVAALKEKV AALKEKVAAL KE

The third polypeptide chain of such a DART-A w/Fc version 1 will comprise the CH2 and CH3 Domains of an IgG Fc Domain. An exemplary polypeptide that is composed of Peptide 1 (DKTHTCPPCP; SEQ ID NO:29) and the CH2 and CH3 Domains of an Fc Domain (SEQ ID NO:26, wherein X is K) and has the sequence of

SEQ ID NO: 30:
DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GEYPSDIAVE
WESNGQPENN YKTTPPVLDS DGSFFLVSKL TVDKSRWQQG
NVFSCSVMHE ALHNRYTQKS LSLSPGK

Additional CD123×CD3 bispecific diabodies comprising alternative optimized anti-CD3 binding domains are provided in WO 2019/160904. In particular, such diabodies comprise the VH_{CD123} Domain of SEQ ID NO:6 and the VL_{CD123} Domain is SEQ ID NO:10 and further comprise an Fe Region.

III. Pharmaceutical Formulations

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a prophylactically or therapeutically effective amount of a CD123×CD3 bispecific binding molecule and a pharmaceutically acceptable carrier.

Exemplary pharmaceutical formulations comprise a CD123×CD3 bispecific binding molecule and an aqueous stabilizer and, optionally, a pharmaceutically acceptable carrier.

As used herein, the term “pharmaceutically acceptable carrier” is intended to refer to a diluent, adjuvant (e.g., Freund's adjuvant (complete and incomplete)), excipient, or vehicle that is approved by a regulatory agency or listed in the U.S. Pharmacopeia or in another generally recognized pharmacopeia as being suitable for delivery into animals, and more particularly, humans. Such pharmaceutical carriers can be sterile liquids, such as water and oils, including those of petroleum, animal, vegetable or synthetic origin, such as peanut oil, soybean oil, mineral oil, sesame oil and the like. Water may be used as carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a liquid formulation, as a dry lyophilized powder or water free concentrate in a hermetically sealed container such as a vial, an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The invention also provides a pharmaceutical pack or kit comprising one or more containers containing a CD123×CD3 bispecific binding molecule alone or with a stabilizer and/or a pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

IV. Kits

The present invention provides kits that comprise a CD123×CD3 bispecific binding molecule, instructional material (for example, relating to storage, dosage, indications, side effects, counter-indications, etc.), and optionally a stabilizer and/or carrier that can be used in the above methods. In such kits, the CD123×CD3 bispecific binding molecule may be packaged in a hermetically sealed container such as an ampoule, a vial, a sachette, etc. that may indicate the quantity of the molecule contained therein. The container may be formed of any pharmaceutically acceptable material, such as glass, resin, plastic, etc. The CD123×CD3 bispecific binding molecule of such kit may be supplied as a liquid solution, a dry sterilized lyophilized powder or a water-free concentrate in a hermetically sealed container that can be reconstituted, e.g., with water or saline to the appropriate concentration for administration to a subject. Such liquid or lyophilized material should be stored at between 2 and 8° C. in its original container and the material should be administered within about 24 hours, with about 12 hours, within about 6 hours, within about 5 hours, within about 3 hours, or within about 1 hour after being reconstituted. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic. In other embodiments, the prophylactic or therapeutic agent is a biological or hormonal therapeutic. The kit can further comprise instructions for use, or other printed information.

Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

V. Methods of Administration

The CD123×CD3 bispecific binding molecule pharmaceutical formulations of the present invention may be provided for the treatment, prophylaxis, and amelioration of one or more symptoms associated with a disease, disorder or infection by administering to a subject an effective amount of a molecule of the invention, or a pharmaceutical composition comprising a fusion protein or a conjugated molecule of the invention. In a one aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a particular embodiment, the subject or patient is a human.

Methods of administering a CD123×CD3 bispecific binding molecule pharmaceutical formulation of the invention include, but are not limited to, parenteral administration (e.g., intradermal, intramuscular, intraperitoneal, intravenous and subcutaneous). In a specific embodiment, the CD123×CD3 bispecific binding molecules are administered intravenously. The compositions may be administered by any convenient route, for example, by infusion, and may be administered together with other biologically active agents.

Administration by infusion is maybe accomplished using an infusion pump. “Infusion pumps” are medical device that deliver fluids into a patient's body in a controlled manner, especially at a defined rate and for a prolonged period of time. Infusion pumps may be powered mechanically, but are more typically electrically powered. Some infusion pumps are “stationary” infusion pumps, and are designed to be used at a patient's bedside. Others, called “ambulatory” infusion pumps, are designed to be portable or wearable. A “syringe” pump is an infusion pump in which the fluid to be delivered is held in the reservoir of a chamber (e.g., a syringe), and a moveable piston is used to control the chamber's volume and thus the delivery of the fluid. In an “elastomeric” infusion pump, fluid is held in a stretchable balloon reservoir, and pressure from the elastic walls of the balloon drives fluid delivery. In a “peristaltic” infusion pump, a set of rollers pinches down on a length of flexible tubing, pushing fluid forward. In a “multi-channel” infusion pump, fluids can be delivered from multiple reservoirs at multiple rates. A “smart pump” is an infusion pump that is equipped a computer-controlled fluid delivery system so as to be capable of alerting in response to a risk of an adverse drug interaction, or when the pump's parameters have been set beyond specified limits. Examples of infusion pumps are well-known, and are provided in, for example, [Anonymous] 2002 “General-Purpose Infusion Pumps,” Health Devices 31(10):353-387; and in U.S. Pat. Nos. 10,029,051, 10,029,047, 10,029,045, 10,022,495, 10,022,494, 10,016,559, 10,006,454, 10,004,846, 9,993,600, 9,981,082, 9,974,901, 9,968,729, 9,931,463, 9,927,943, etc.

In certain embodiments, the CD123×CD3 bispecific binding molecule pharmaceutical formulations of the invention be administered by infusion facilitated by one or more ambulatory pumps, so that the patient will be ambulatory during the therapeutic regimen. In certain embodiments, the CD123×CD3 bispecific binding molecule pharmaceutical formulations of the invention be administered by continuous infusion. In a specific embodiment, a 7-day continuous infusion regimen comprises a treatment dosage of about 30 ng/kg patient weight/day for 3 days followed by a treatment dosage of about 100 ng/kg/day for 4 days (for example, a treatment dosage of 30 ng/kg patient weight/day for 3 days followed by a treatment dosage of 100 ng/kg/day for 4 days; etc.). In another specific embodiment, a 7-day continuous infusion regimen comprises a treatment dosage of about 30 ng/kg patient weight/day for 1 day, followed by a treatment dosage of about 60 ng/kg patient weight/day for 1 day, followed by a treatment dosage of about 100 ng/kg/day for 1 day, followed by a treatment dosage of about 200 ng/kg/day for 1 day, followed by a treatment dosage of about 300 ng/kg/day for 1 day, followed by a treatment dosage of about 400 ng/kg/day for 1 day, followed by a treatment dosage of about 500 ng/kg/day for 1 day. In certain embodiments, such 7-day continuous infusion regiment is followed by a 21-day continuous infusion regiment in which a treatment dosage of 500 ng/kg/day is administered every day for 21 days. In certain embodiments, such 21-day continuous infusion regiment is followed by a 21-day continuous infusion regiment in which a treatment dosage of about 500 ng/kg/day is administered during days 1-4 of each week of such 21-day regiment and during days 5-7 of each week no treatment dosage is administered.

In any of the above-described courses of treatment, the proportion of CD8+T-lymphocytes in the tumor microenvironment may additionally be monitored. Such monitoring may occur prior to the administration of the CD123×CD3 bispecific binding molecule, during the course of CD123×CD3 binding molecule therapy, and/or after the conclusion of a cycle of CD123×CD3 binding molecule therapy.

VI. Uses of the Compositions of the Invention

The CD123×CD3 bispecific binding molecules of the invention may be used to treat any disease or condition associated with or characterized by the expression of CD123. In particular, the CD123×CD3 bispecific binding molecules of the invention may be used to treat hematologic malignancies. The CD123×CD3 bispecific binding molecules of the invention are particularly suitable for use in the treatment of hematologic malignancies, including chemo-refractory hematologic malignancies. As used herein, a chemo-refractory hematologic malignancy is a hematologic malignancy that is refractory to two or more induction attempts, a first CR of less than 6 months, or a failure after two or more cycles of treatment with a hypomethylating agent).

Thus, without limitation, such molecules may be employed in the diagnosis or treatment of acute myeloid leukemia (AML) (including primary chemo-refractory AML), chronic myelogenous leukemia (CML), including blastic crisis of CML and Abelson oncogene-associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), including Richter's syndrome or Richter's transformation of call, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma. The CD123×CD3 bispecific binding molecules of the invention may additionally be used in the manufacture of medicaments for the treatment of the above-described conditions.

The CD123×CD3 bispecific binding molecules of the invention are particularly suitable for use in the treatment of acute myeloid leukemia (AML, including primary chemo-refractory acute myeloid leukemia), hematologic myelodysplastic syndrome (MDS), blastic plasmacytoid dendritic cell neoplasm (BPDCN), non-Hodgkin's lymphoma (NHL), or acute T lymphoblastic leukemia (T-ALL).

### EXAMPLES

Having now generally described the invention, the same will be more readily understood through reference to the following examples, which are provided by way of illustration and are not intended to be limiting of the present invention unless specified.

### Example 1

### Gene Expression Signatures of Patient Populations Particularly Suitable for Treatment with a CD123×CD3 Bispecific Binding Molecule of the Invention

In order to demonstrate a correlation between the expression patterns of the genes of patients having a hematologic malignancy, particularly AML, and the favorable outcome of CD123×CD3 bispecific binding molecule therapy, RNA was isolated from bone marrow (“BM”) samples obtained from patients with individual patient consent from patients with relapsed or refractory AML enrolled in a phase ½ clinical trial of flotetuzumab (NCT #02152956, an exemplary CD123×CD3 bispecific binding molecule).

The expression of 770 immune-related genes, including predefined immune gene signature scores examined in baseline bone marrow samples of a subgroup of patients treated with the exemplary CD123×CD3 bispecific molecule flotetuzumab. Briefly, The NanoString PanCancer IO360™ assay was used to interrogate the expression of 770 genes, including the abundance of 14 immune cell types and 32 immuno-oncology signatures in bone marrow samples of a subgroup of patients (n=38) with relapsed/refractory AML treated with flotetuzumab at the recommended phase 2 dose (RP2D; 38 bone marrow samples collected at baseline and 34 bone marrow samples collected on treatment (post-cycles 1 [n=25] and 2 [n=9]). IO 360 gene counts were generated using the nCounter® system (NanoString Technologies, Inc.) essentially as follows: RNA (˜100 ng per sample) was extracted from unfractionated bone marrow aspirates, and was incubated with reporter and capture probe mix for hybridization. Transcript counts were analyzed on the nCounter FLEX analysis system using the high-resolution setting. Reporter code count (RCC) output files are used to calculate gene signature scores. Signature scores for predefined signatures described by NanoString were calculated as pre-defined linear combinations (weighted averages) of biologically relevant gene sets essentially as described in WO 2020/092404 and Vadakekolathu J, et al. (2020) “Immune Landscapes Predict Chemotherapy Resistance And Immunotherapy Response In Acute Myeloid Leukemia,” Sci. Transl. Med. 12(546).eaaz0463. In addition, a ranked gene list (χ² values) was generated using Orange3 software package (Version 3.25.0). Unsupervised hierarchical clustering (Euclidean distance, complete linkage).

As previously reported patients with primary induction failure (PIF)/early relapse (ER) showed higher immune infiltration relative to those exhibiting late relapse (LR). PD-L1 and inflammatory chemokine scores were higher in PIF/ER and HMA-treated patients compared with LR. The tumor inflammation signature score (TIS) correlated with the antigen processing machinery and inflammatory chemokine scores (P<0.0001), suggesting the occurrence of antigen presentation and T cell chemoattraction in highly T cell-inflamed samples (Vadakekolathu J, et al. (2020) “Immune Landscapes Predict Chemotherapy Resistance And Immunotherapy Response In Acute Myeloid Leukemia.” Sci. Transl. Med. 12(546):eaaz0463).

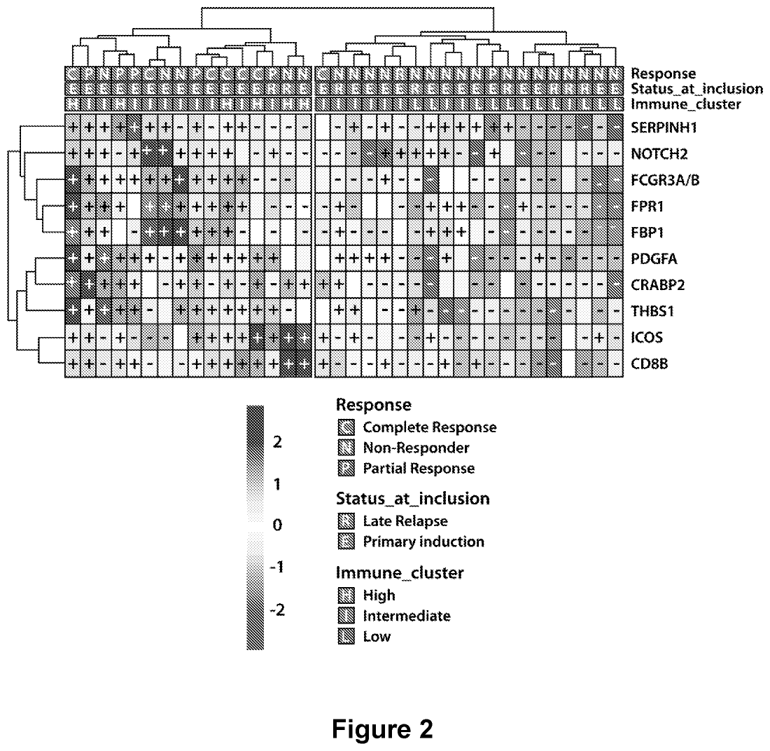

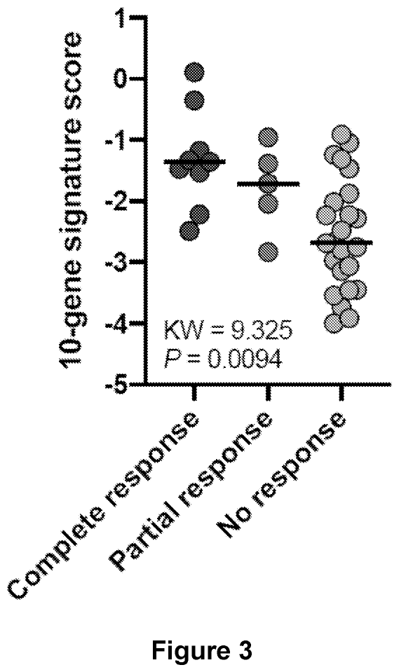

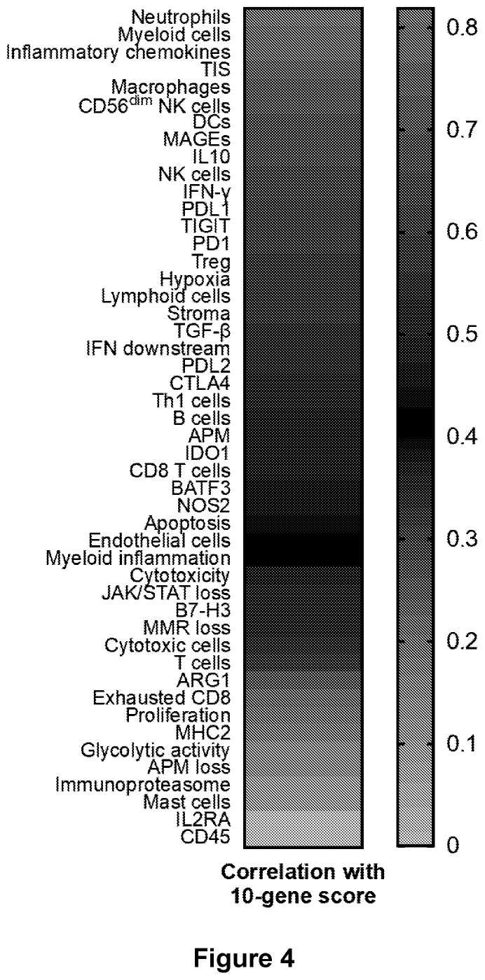

Further analysis was performed by ranking the 770 immune-related genes in the gene expression panel. This ranking identified a parsimonious expression signature encompassing the top 10 genes associated with complete response to the exemplary CD123×CD3 bispecific molecule flotetuzumab, defined as either complete remission (CR), complete remission with incomplete hematopoietic recovery (CRi) or complete remission with partial hematopoietic recovery (CRh). The 10 identified genes: CD8B, CRABP2, FCGR3A/B, FBP1, FPR1, ICOS, NOTCH2, PDGFA, SERPINH1, and THBS1 are listed in Table 1 above. Table 1 also provides a representative, non-limiting NCBI Accession Number for each gene. A heatmap showing the expression of these 10 genes associated with complete response to the exemplary CD123×CD3 bispecific molecule flotetuzumab is provided in FIG. 2. The newly identified 10-gene signature score was calculated as the average sum of gene expression across the patient cohort. The expression of this 10-gene signature was higher in patients with PIF/ER at time of study enrollment relative to LR and in those with a high or intermediate immune cluster score. FIG. 3 plots the 10-gene signature scores according to patient response (complete response, partial response, or no response) and shows patients exhibiting an antileukemic response has higher 10-gene signature scores. As shown in the heatmap presented in FIG. 4, this 10-gene signature score correlated with the degree of bone marrow immune infiltration as measured determined by expression of predefined immune gene signature scores (NanoString), including neutrophil, macrophage and myeloid cell types, and with inflammatory chemokines and other signature scores that reflect a T cell-inflamed, IFN-7-driven TME. Without being bound by any particular mechanism it is noted that genes in the signature include CD8B, immune checkpoint ICOS and NOTCH2, all of which reflect a T cell-driven and highly immunosuppressed tumor microenvironment that could be re-invigorated by CD123×CD3 bispecific binding molecules such as flotetuzumab. In this respect, increased Notch signaling has been correlated with enhanced CD8 T-cell infiltration in patients with colorectal carcinoma and with inhibited T-cell responses. Furthermore, Notch2, but not Notch1 signaling, is critically required for the generation of cytotoxic T lymphocytes with anti-tumor activity in experimental models of lymphoma and acts as a transcriptional activator of granzyme B.

Analysis of functional protein association networks was performed using STRING (string-db.org). These data indicated that the 10 genes associated with complete response were enriched in ontologies and pathways related to antigen binding and processing, VEGF-activated receptor activity, Notch signaling, micro-RNA regulation in cancer and T helper type 1 (Th1) and Th2 differentiation (Table 5). These data support the potential role of enhanced and sustained antigen presentation in the TME in promoting anti-leukemia responses from CD123×CD3 bispecific binding molecules such as flotetuzumab.

TABLE 5
Gene Ontologies (GO) and KEGG Pathways Captured by the Top 10
Genes Associated with Complete Response to Flotetuzumab

| | | Count in | |
|---|---|---|---|
| GO term | Description | Gene Set | FDR |
| GO: 0005102 | Signaling receptor binding | 25 of 1513 | 1.63 × 10⁻¹² |
| GO: 0042605 | Peptide antigen binding | 7 of 22 | 1.00 × 10⁻¹⁰ |
| GO: 0003823 | Antigen binding | 8 of 56 | 3.98 × 10⁻¹⁰ |
| GO: 0019838 | Growth factor binding | 9 of 126 | 4.05 × 10⁻⁹ |
| GO: 0005518 | Collagen binding | 7 of 61 | 2.31 × 10⁻⁸ |
| GO: 0005161 | Platelet-derived growth factor | 5 of 15 | 6.26 × 10⁻⁸ |
| | receptor binding | | |
| GO: 0005515 | Protein binding | 38 of | 1.25 × 10⁻⁷ |
| | | 326605 | |
| GO: 0005021 | Vascular endothelial growth | 4 of 7 | 4.24 × 10⁻⁷ |
| | factor-activated receptor activity | | |
| GO: 0046977 | TAP binding | 4 of 7 | 4.24 × 10⁻⁷ |
| GO: 0044877 | Protein-containing complex | 15 of 968 | 4.26 × 10⁻⁷ |
| | binding | | |
| GO: 0048407 | Platelet-derived growth factor | 4 of 11 | 1.26 × 10⁻⁶ |
| | binding | | |
| hsa05165 | Human papillomavirus infection | 20 of 317 | 1.56 × 10⁻²⁰ |
| hsa04330 | Notch signaling pathway | 10 of 48 | 1.49 × 10⁻¹⁴ |
| hsa05166 | HTLV-I infection | 14 of 250 | 1.43 × 10⁻¹³ |
| hsa05206 | MicroRNAs in cancer | 12 of 149 | 2.12 × 10⁻¹³ |
| hsa04658 | Th1 and Th2 cell differentiation | 10 of 888 | 1.49 × 10⁻¹² |
| hsa04612 | Antigen processing and | 9 of 66 | 5.40 × 10⁻¹² |
| | presentation | | |
| hsa04145 | Phagosome | 10 of 145 | 1.12 × 10⁻¹⁰ |
| hsa05200 | Pathways in cancer | 151 of 515 | 6.68 × 10⁻¹⁰ |
| hsa04015 | Rapl signaling pathway | 10 of 203 | 1.82 × 10⁻⁹ |
| hsa04514 | Cell adhesion molecules | 9 of 139 | 1.82 × 10⁻⁹ |
| | (CAMs) | | |
| hsa05330 | Allograft rejection | 6 of 35 | 9.90 × 10⁻⁹ |

FDR = false discovery rate

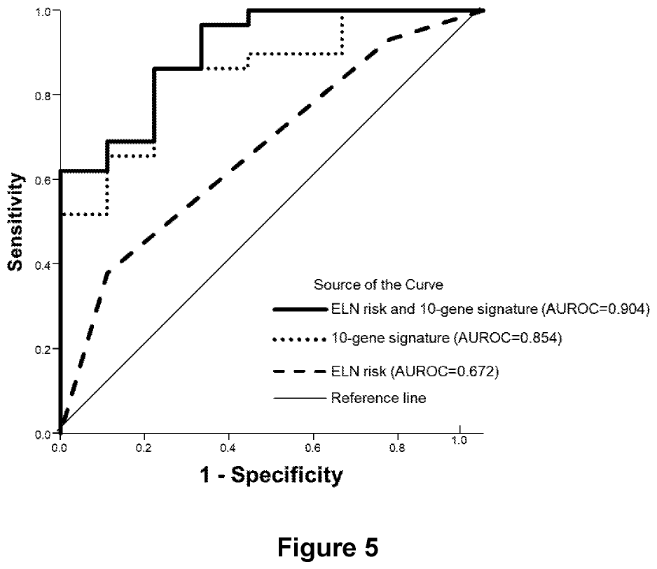

AUROC curves measuring the predictive ability of European Leukemia-Net (ELN) risk disease status (Dohner H, et al., 2017, “Diagnosis and management of AML in adults: 2017 ELN recommendations from an international expert panel.” Blood 129(4): 424-47), at study entry and the 10-gene signature score for anti-leukemic activity of the exemplary CD123×CD3 bispecific binding molecule flotetuzumab, either individually or in combination, are shown in FIG. 5, standard errors and confidence intervals are provided in Table 6. Notably, the 10-gene signature score had an AUROC value of 0.854 when considered alone and of 0.904 when in conjunction with the ELN risk category, compared with 0.672 for the ELN risk category alone. In addition, treatment with the CD123×CD3 bispecific binding molecule flotetuzumab modulated the TME as suggested by heightened levels of immune infiltration, PD-L1 expression, antigen presentation and IFN-γ signaling gene signatures.

TABLE 6
AUROC Curve Analysis

| | | | | 95% CI | |
|---|---|---|---|---|---|
| | | | | Lower | Upper |
| Variable | Area | S.E. | P value | bound | bound |
| ELN risk | 0.672 | 0.10 | 0.122 | 0.476 | 0.869 |
| 10-gene signature score | 0.854 | 0.067 | 0.001 | 0.724 | 0.985 |
| ELN risk + 10-gene | 0.904 | 0.055 | 0.000 | 0.797 | 1.000 |
| signature score | | | | | |

SE = standard error;
CI = confidence interval.
AUROC = 1.0 would denote perfect prediction and AUROC = 0.5 would denote no predictive ability. AUROC curves were estimated using the SPSS software package, as previously published (Vadakekolathu J. et al. (2020) “Immune Landscapes Predict Chemotherapy Resistance And Immunotherapy Response In Acute Myeloid Leukemia,” Sci. Transl. Med. 12(546):eaaz0463; Wagner S, et al. (2019) “A Parsimonious 3-Gene Signature Predicts Clinical Outcomes In An Acute Myeloid Leukemia Multicohort Study,” Blood Adv. 3(8): 1330-1346)

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of determining whether a patient would be a suitable responder to the use of a CD123×CD3 bispecific molecule to treat a hematologic malignancy, wherein said method comprises:
(a) evaluating the expression of one or more target genes in a cellular sample from said patient prior to the administration of said CD123×CD3 bispecific molecule, relative to the expression of one or more target and/or reference genes; and
(b) identifying the patient as a suitable responder for treatment with a CD123×CD3 bispecific molecule if the expression of said one or more target genes is found to be increased relative to said expression of said one or more target and/or reference genes, wherein said one or more target genes are selected from the group consisting of: SERPHINH1, NOTCH2, FCGR3A/B, FPR1, FBP1, PDGFA, CRABP2, THBS1, ICOS and CD8B.

2. The method of claim 1, wherein said method evaluates:
(i) the expression of one or more target genes; and
(ii) one or more reference genes whose expression is not characteristically associated with said hematologic malignancy.

3. The method of any one of claims 1-2, wherein said method comprises evaluating the expression of said one or more target genes relative to the baseline expression of said one or more reference genes of said patient.

4. The method of any one of claims 1-3, wherein said method comprises evaluating the expression of said one or more target genes of said patient relative to the expression of said one or more target genes of:
(a) an individual who is suffering from said hematologic malignancy, or of a population of such individuals; or
(b) an individual who did not successfully respond to the use of a CD123×CD3 bispecific molecule to treat said hematologic malignancy, or of a population of such individuals; or
(c) an individual who successfully responded to the use of a CD123× CD3 bispecific molecule to treat said hematologic malignancy, or of a population of such individuals.

5. The method of any one of claims 1-4, wherein the relative expression level of said one or more target genes in said population is established by averaging the gene expression level in cellular samples obtained from said population of individuals.

6. The method of any one of claims 1-5, wherein said patient exhibits an expression level of at least one of said target genes:
(a) that is greater than the first quartile of the expression levels of said target gene in a population of individuals who are suffering from said hematologic malignancy; or
(b) that is greater than the first quartile of the expression levels of said target gene in a population of individuals who did not successfully respond to a treatment for said hematologic malignancy that used a CD123×CD3 bispecific molecule; or
(c) that is within at least the first quartile of the expression levels of said target gene in a population of individuals who successfully responded to a treatment for said hematologic malignancy that used a CD123×CD3 bispecific molecule.

7. The method of any one of claims 1-6, wherein said method further comprises administering a treatment dosage of said CD123×CD3 bispecific molecule to said patient if said patient is determined to be a suitable responder to such treatment, and wherein said administration of said CD123×CD3 bispecific molecule stimulates the killing of cells of said hematologic malignancy in said patient.

8. A method of treating a hematologic malignancy, wherein said method comprises:
(a) employing the method of any one of claims 1-6 to determine whether a patient would be a suitable responder to the use of a CD123×CD3 bispecific molecule to treat said hematologic malignancy;
(b) administering a treatment dosage of said CD123×CD3 bispecific molecule to said patient if said patient is determined to be a suitable responder to such treatment;
wherein said administration of said CD123×CD3 bispecific molecule stimulates the killing of cells of said hematologic malignancy in said patient.

9. The method of any one of claims 1-8, wherein said cellular sample is a bone marrow sample.

10. The method of any one of claims 1-9, wherein said evaluation of expression or said determination of whether said patient would be a suitable responder to the use of a CD123×CD3 bispecific molecule to treat a hematologic malignancy is performed by:
(a) determining the gene expression levels for each target gene in one or more cellular sample(s) using a gene expression platform; and
(b) comparing said target gene expression levels to the expression levels of one or more reference genes.

11. The method of any one of claims 1-10, wherein said evaluation of expression or said determination of whether said patient would be a suitable responder to the use of a CD123×CD3 bispecific molecule to treat a hematologic malignancy is performed by:
(a) measuring the raw RNA levels in for each target gene in one more cellular sample using a gene expression platform, wherein the gene expression platform comprises a reference gene set of housekeeping genes, and
(b) assigning a relative expression value, for each of the measured raw RNA levels for the target genes using the measured RNA levels of the internal reference genes.

12. The method of any one of claims 1-11, wherein said one or more reference genes comprise one or more of: ABCF1, G6PD, NRDE2, OAZ1, POLR2A, SDHA, STK11IP, TBC1D10B, TBP, and UBB.

13. The method of any one of claims 1-12, wherein a gene signature score is determined for said one or more target genes.

14. The method of claim 13, wherein said gene signature score is determined by a process comprising:
(a) measuring the raw RNA levels for each target gene in one more cellular sample using a gene expression platform comprising a reference gene set of housekeeping genes,
(b) normalizing each of the measured raw RNA levels to the geometric mean of said housekeeping genes, and optionally further normalizing each RNA value to a standard,
(c) log transforming each normalized RNA value,
(d) summing the log transformed RNA values for each target gene in the signature, and
(e) dividing the sum of the normalized log transformed RNA values by the number of target genes in the signature, to generate a gene signature score.

15. The method of any one of claims 13-14, wherein a patient gene signature score that:
(a) is greater than the first quartile of scores for said gene signature calculated from the expression levels of one or more of said target genes in a population of individuals who are suffering from said hematologic malignancy; or
(b) is greater than the first quartile of scores for said gene signature calculated from the expression levels of one or more of said target genes in a population of individuals who did not successfully respond to a treatment for said hematologic malignancy that used a CD123×CD3 bispecific molecule; or
(c) is within at least the first quartile of the scores for said gene signature calculated from the expression levels of one or more of said target genes in a population of individuals who successfully responded to a treatment for said hematologic malignancy that used a CD123×CD3 bispecific molecule,
is indicative of a more favorable patient response to treatment with said CD123×CD3 bispecific molecule.

16. The method of any one of claims 1-15, wherein said CD123×CD3 bispecific molecule is a bispecific antibody or bispecific molecule comprising an scFv.

17. The method of claim 16, wherein said CD123×CD3 bispecific molecule is JNJ-63709178, XmAb14045 or APVO436.

18. The method of any one of claims 1-15, wherein said CD123×CD3 bispecific molecule is a covalently bonded bispecific diabody having two, three, or four polypeptide chains.

19. The method of claim 18, wherein said diabody comprises:
(a) a first polypeptide chain having the amino acid sequence of SEQ ID NO:21; and
(b) a second polypeptide chain having the amino acid sequence of SEQ ID NO:23; and
wherein the first and said second polypeptide chains are covalently bonded to one another by a disulfide bond.

20. The method of any one of claims 1-19, wherein said hematologic malignancy of said patient is selected from the group consisting of: acute myeloid leukemia (AML), chronic myelogenous leukemia (CML), blastic crisis of CML, Abelson oncogene-associated with CML (Bcr-ABL translocation), myelodysplastic syndrome (MDS), acute B lymphoblastic leukemia (B-ALL), acute T lymphoblastic leukemia (T-ALL), chronic lymphocytic leukemia (CLL), Richter's syndrome, Richter's transformation of CLL, hairy cell leukemia (HCL), blastic plasmacytoid dendritic cell neoplasm (BPDCN), non-Hodgkin's lymphoma (NHL), including mantle cell lymphoma (MCL) and small lymphocytic lymphoma (SLL), Hodgkin's lymphoma, systemic mastocytosis, and Burkitt's lymphoma.

21. The method of claim 21, wherein said hematologic malignancy of said patient is AML.

22. The method of any one of claims 1-21, wherein said hematologic malignancy of said patient is refractory to chemotherapy.

23. The method of any one of claims 1-22, wherein said treatment dosage of said CD123×CD3 bispecific molecule includes at least one dose selected from the group consisting of about 30, about 60, about 100, about 200, about 300, about 400 and about 500 ng/kg patient weight/day.

24. The method of any one of claims 1-23, wherein said treatment dosage is administered by continuous infusion.

25. The method of any one of claims 1-24, wherein said patient is a human patient.
